(19) United States
(12) Patent Application Publication
(10) Pub. No.: US 2009/0124635 A1
(43) Pub. Date: May 14, 2009

(54) CHEMICAL COMPOUNDS

(75) Inventors: Christopher Gordon Barber, Kent (GB); David Clive Blakemore, Kent (GB); David Cameron Pryde, Kent (GB); James Welsh Auld Kinnaird, Kent (GB)

Correspondence Address: PFIZER INC;Steve T. Zelson, 150 EAST 42ND STREET, 5TH FLOOR - STOP 49, NEW YORK, NY, 10017-5612, US

(73) Assignee: PFIZER INC., Sandwich, Kent (GB)

(21) Appl. No.: 11/813,852

(22) PCT Filed: Jan. 12, 2006
(86) PCT No.: PCT/IB06/00170

§ 371 Date: Oct. 17, 2008

Related U.S. Application Data
(60) Provisional application No. 60/658,108, filed on Mar. 2, 2005.

(30) Foreign Application Priority Data
Jan. 20, 2005 (GB) 0501188.7

(51) Int. Cl. A61K 31/4545; C07D 401/14; A61K 31/506; A61P 31/12; A61P 37/00; A61P 29/00; A61P 31/18; C07D 405/14

(52) U.S. Cl. 514/256; 546/187; 544/335; 546/112; 514/299; 514/316

(57) ABSTRACT

The present invention provides compounds of formula (I) wherein R¹, R², R³, R⁴, R⁵, m and n are as defined hereinabove. The compounds of the present invention are modulators, especially antagonists, of the activity of chemokine CCR5 receptors. Modulators of the CCR5 receptor may be useful in the treatment of various inflammatory diseases and conditions, and in the treatment of infection by HIV and genetically related retroviruses.

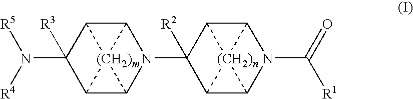

This invention relates to piperidine derivatives, to processes for their preparation, to compositions containing them and to their use.

More particularly, the present invention relates to the use of piperidine derivatives in the treatment of a variety of disorders, including those in which the modulation, in particular antagonism, of chemokine CCR5 receptors is implicated. Accordingly, compounds of the invention are useful in the treatment of HIV, such as HIV-1, and genetically related retroviral infections (and the resulting acquired immune deficiency syndrome, AIDS), inflammatory diseases, autoimmune diseases and pain.

The name “chemokine”, is a contraction of “chemotactic cytokines”. The chemokines comprise a large family of proteins which have in common important structural features and which have the ability to attract leukocytes. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory and infectious diseases, agents which are active in modulating, preferably antagonising, the activity of chemokines and their receptors, are useful in the therapeutic treatment of such inflammatory and infectious diseases.

The chemokine receptor CCR5 is of particular importance in the context of treating inflammatory and infectious diseases. CCR5 is a receptor for chemokines, especially for the macrophage inflammatory proteins (MIP) designated MIP-1α and MIP-1β, and for a protein which is regulated upon activation and is normal T-cell expressed and secreted (RANTES).

We have now found a group of compounds that are both potent and selective modulators, in particular antagonists, of the CCR5 receptor.

According to a first aspect of the present invention, there is provided a compound of formula (I)

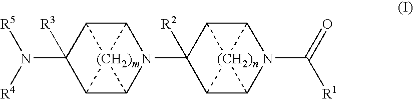

or a pharmaceutically acceptable salt, solvate or derivative thereof, wherein:

R¹ is phenyl; napthyl; or a 5 to 10-membered aromatic heterocycle; wherein said heterocycle contain one to three heteroatoms selected from N, O or S; and wherein the said phenyl, napthyl and heterocycle are substituted by 0 to 3 atoms or groups selected from C₁₋₆ alkyl, C₃₋₇ cycloalkyl, C₁₋₆ alkoxy, C₁₋₆alkoxyC₁₋₆ alkyl, halogen, C₁₋₆ haloalkyl, OH, CN, NR⁸R⁹, COR⁸, CO₂R⁸, CONR⁸R⁹, phenyl, imidazolyl, or, wherein R¹ is a heterocycle, oxo;

R² and R³ are independently H or C₁₋₆ alkyl;

R⁴ is benzyl, pyridylmethyl or pyrimidinylmethyl, wherein the said benzyl, pyridylmethyl and pyrimidinylmethyl are substituted by 0 to 3 atoms or groups selected from alkyl, C₃₋₇ cycloalkyl, C₁₋₆ alkoxy, C₁₋₆ alkoxyC₁₋₆ alkyl, halogen, CM haloalkyl, OH, CN, NR⁸R⁹, COR⁸, CO₂R⁸, CONR⁸R⁹, phenyl or imidazolyl;

R⁵ is COR⁶ or SO₂R⁷;

R⁶ is C₁₋₆ alkyl, C₃₋₇ cycloalkyl, C₁₋₆ alkoxy, C₃₋₇ cycloalkyC₁₋₃ alkyl, C₁₋₆ alkoxyC₁₋₆ alkyl, tetrahydrofuryl or tetrahydropyranyl; wherein the said C₁₋₆ alkyl, C₃₋₇ cycloalkyl, C₁₋₆ alkoxy and C₁₋₆ alkoxyC₁₋₆ alkyl are substituted by 0 to 3 atoms or groups selected from halogen, NR⁸R⁹, C₁₋₆ alkoxy or OH;

R⁷ is C₁₋₆ alkyl;

R⁸ and R⁹ are independently H or C₁₋₆ alkyl; or, when R⁸ and R⁹ are both attached to the same N atom, NR⁸R⁹ may also represent a 5 to 7 membered, saturated, partially unsaturated or aromatic, heterocycle containing from 0 to 2 additional heteroatoms selected from O, N or S;

m is 0, 1, 2 or 3;

n is 0, 1, 2 or 3;

“-----” represents an optionally present C—C bond such that, when m or n= 1, 2 or 3, any two of the bonds are present per piperidine ring to form an alkylene bridge.

The term “alkyl” as a group or part of a group includes straight chain and branched groups. Examples of alkyl include methyl, ethyl, n-propyl, i-propyl, n-butyl, i-butyl, sec-butyl and t-butyl. The term “C₃-C₇ cycloalkyl” means cyclopropyl, cyclobutyl, cyclopentyl, cyclohexyl or cycloheptyl. The term halogen means fluoro, chloro, bromo or iodo. The term “C₁₋₆ haloalkyl” means C₁₋₆ alkyl substituted by one or more halogen atoms.

In one embodiment, R¹ is phenyl, pyridyl, pyrimidyl, pyridyl N-oxide or pyrimidyl N-oxide; wherein the said phenyl, pyridyl, pyrimidyl, pyridyl N-oxide and pyrimidyl N-oxide are substituted by 0 to 3 atoms or groups selected from C₁₋₆ alkyl, C₃₋₇ cycloalkyl, C₁₋₆ alkoxy, C₁₋₃ alkoxyC₁₋₃ alkyl, halogen, C₁₋₆ haloalkyl, OH, CN, NR⁸R⁹, COR⁸, CO₂R⁸, CONR⁸R⁹, phenyl or imidazolyl.

In a further embodiment, R¹ is phenyl, pyridyl, pyrimidyl, pyridyl N-oxide or pyrimidyl N-oxide; wherein the said phenyl, pyridyl, pyrimidyl, pyridyl N-oxide and pyrimidyl N-oxide are substituted by 0 to 3 atoms or groups selected from C₁₋₆ alkyl or halogen.

In yet a further embodiment, R¹ is phenyl, pyridyl, pyrimidyl, pyridyl N-oxide or pyrimidyl N-oxide; wherein the said phenyl, pyridyl, pyrimidyl, pyridyl N-oxide and pyrimidyl N-oxide are substituted by 0 to 3 atoms or groups selected from C₁₋₆ alkyl or halogen.

In yet a further embodiment, R¹ is phenyl, pyridyl, pyrimidyl, pyridyl N-oxide or pyrimidyl N-oxide; wherein the said phenyl, pyridyl, pyrimidyl, pyridyl N-oxide and pyrimidyl N-oxide are substituted by 0 to 3 atoms or groups selected from methyl or chlorine.

In yet a further embodiment, R¹ is phenyl, pyridyl, pyrimidyl, pyridyl N-oxide or pyrimidyl N-oxide; wherein the said phenyl, pyridyl, pyrimidyl, pyridyl N-oxide and pyrimidyl N-oxide are substituted by 2 atoms or groups selected from methyl or chlorine.

In yet a further embodiment, R² and R³ are independently H or C₁₋₃ alkyl.

In yet a further embodiment, R² and R³ are independently H or methyl.

In yet a further embodiment, R⁴ is benzyl substituted by 0 to 3 atoms or groups selected from C₁₋₆ alkyl, C₃₋₇ cycloalkyl, C₁₋₆ alkoxy, C₁₋₃ alkoxyC₁₋₃ alkyl, halogen, C₁₋₆ haloalkyl, OH, CN, NR⁸R⁹, COR⁸, CO₂R⁸, CONR⁸R⁹, phenyl or imidazolyl.

In yet a further embodiment, R⁴ is benzyl substituted by 0 to 3 atoms or groups selected from C₁₋₃ alkyl, C₁₋₃ alkoxy, halogen, or C₁₋₃ haloalkyl.

In yet a further embodiment, R⁴ is benzyl substituted by 0 to 3 atoms or groups selected from methyl, methoxy, fluorine, chlorine or CF₃.

In yet a further embodiment, R⁵ is COR⁶.

In yet a further embodiment, R⁵ is SO₂R⁷.

In yet a further embodiment, R⁶ is C₁₋₆ alkyl, C₃₋₆ cycloalkyl, C₃₋₅ cycloalkyC₁₋₂ alkyl, C₁₋₃ alkoxy, C₁₋₃ alkoxyC₁₋₃ alkyl, tetrahydrofuryl or tetrahydropyranyl; wherein the said C₁₋₃ alkyl, C₃₋₆ cycloalkyl, C₃₋₅ cycloalkyC₁₋₂ alkyl, C₁₋₃ alkoxy and C₁₋₃ alkoxyC₁₋₃ alkyl are substituted by 0 to 3 atoms or groups selected from halogen.

In yet a further embodiment, R⁶ is C₁₋₄ alkyl or C₃₋₆ cycloalkyl; wherein the said C₁₋₃ alkyl and C₃₋₆cycloalkyl are substituted by 0 to 3 atoms selected from halogen.

In yet a further embodiment, R⁷ is C₁₋₃ alkyl.

In yet a further embodiment, R⁷ is methyl.

In yet a further embodiment, R⁸ and R⁹ are independently H or C₁₋₃ alkyl.

In yet a further embodiment, R⁸ and R⁹ are independently H or methyl.

In yet a further embodiment there is provided a compound of formula (Ia)

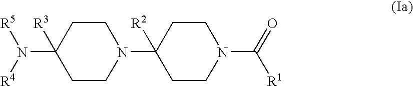

or a pharmaceutically acceptable salt, solvate or derivative thereof, wherein R¹, R², R³, R⁴ and R⁵ are as defined hereinabove with respect to a compound of formula (I), including all combinations of particular described embodiments thereof.

In yet a further embodiment there is provided a compound of formula (Ib)

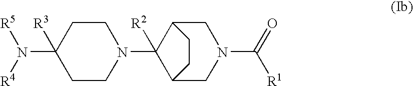

or a pharmaceutically acceptable salt, solvate or derivative thereof, wherein R¹, R², R³, R⁴ and R⁵ are as defined hereinabove with respect to a compound of formula (I), including all combinations of particular described embodiments thereof.

In yet a further embodiment there is provided a compound of formula (Ic)

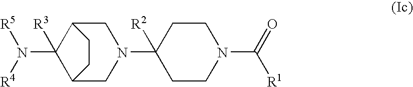

or a pharmaceutically acceptable salt, solvate or derivative thereof, wherein R¹, R², R³, R⁴ and R⁵ are as defined hereinabove with respect to a compound of formula (I), including all combinations of particular described embodiments thereof.

In yet a further embodiment there is provided a compound of formula (Id)

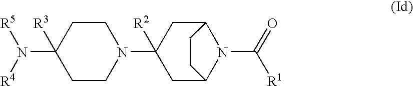

or a pharmaceutically acceptable salt, solvate or derivative thereof, wherein R¹, R², R³, R⁴ and R⁵ are as defined hereinabove with respect to a compound of formula (I), including all combinations of particular described embodiments thereof.

In yet a further embodiment there is provided a compound of formula (Ie)

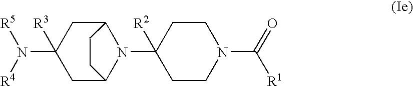

or a pharmaceutically acceptable salt, solvate or derivative thereof, wherein R¹, R², R³, R⁴ and R⁵ are as defined hereinabove with respect to a compound of formula (I), including all combinations of particular described embodiments thereof.

It is to be understood that the invention covers all combinations of embodiments of the invention as described hereinabove, consistent with the definition of compounds of formula (I).

The compounds of the invention include compounds of formula (I) and pharmaceutically acceptable salts, solvates or derivatives thereof (wherein derivatives include complexes, prodrugs and isotopically-labelled compounds, as well as salts and solvates thereof). In a further embodiment, the compounds of the invention are the compounds of formula (I) and pharmaceutically acceptable salts and solvates thereof, in particular the compounds of formula (I). It is to be understood that the aforementioned compounds of the invention include polymorphs and isomers thereof.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002), incorporated herein by reference.

Pharmaceutically acceptable salts of compounds of formula (I) may be prepared by one or more of three methods:

(i) by reacting the compound of formula (I) with the desired acid;
(ii) by removing an acid- or base-labile protecting group from a suitable precursor of the compound of formula (I) or by ring-opening a suitable cyclic precursor, for example, a lactone or lactam, using the desired acid; or
(iii) by converting one salt of the compound of formula (I) to another by reaction with an appropriate acid or by means of a suitable ion exchange column.

All three reactions are typically carried out in solution. The salt may precipitate from solution and be collected by filtration or may be recovered by evaporation of the solvent. The degree of ionisation in the salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

Hereinafter all references to compounds of formula (I) include references to salts, solvates, multi-component complexes and liquid crystals thereof and to solvates, multi-component complexes and liquid crystals of salts thereof.

Certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985). Some examples of prodrugs in accordance with the invention include:
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula I is replaced by (C₁-C₆)alkanoyloxymethyl; and (iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types in accordance with the invention may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include:
(i) where the compound of formula (I) contains a methyl group, an hydroxymethyl derivative thereof (—CH₃->—CH₂OH);
(ii) where the compound of formula (I) contains an alkoxy group, an hydroxy derivative thereof (—OR->—OH);
(iii) where the compound of formula (I) contains a tertiary amino group, a secondary amino derivative thereof (—NR¹R²->—NHR¹ or —NHR²);
(iv) where the compound of formula (I) contains a secondary amino group, a primary derivative thereof (—NHR¹->—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂->COOH).

Compounds of formula (I) may contain one or more asymmetric carbon atoms and therefore exist as two or more stereoisomers. Compounds of formula (I) wherein m or n≠0, i.e., which contain a bridged piperidine ring, can be in either endo- or exo-configuration, and therefore geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, a keto, or oxime group, or so-called valence tautomerism in compounds which contain an aromatic moiety.

Compounds of formula (I) may exhibit atropisomerism, or axial chirality, which occurs when molecules are chiral by virtue of their overall shape rather than having chiral centres. The 3D shape which renders these molecules chiral is maintained as a result of hindered rotation around a bond or bonds. Free rotation about a single covalent bond is impeded sufficiently that interconversion of the stereoisomeric conformations (atropisomers) is slow enough to allow separation and isolation under predetermined conditions. The energy barrier to thermal racemization may be determined by the steric hindrance to free rotation of one or more bonds forming a chiral axis

It follows that a single compound may exhibit more than one type of isomerism.

Included within the scope of the present invention are all stereoisomers of the compounds of formula (I), including all optical isomers, geometric isomers, atropisomers and tautomeric forms as well as compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Endo/exo isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E. L. Eliel (Wiley, New York, 1994).

The present invention also includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

Preferred compounds of formula (I) include the compounds of Examples 1-83; and pharmaceutically acceptable salts, solvates or derivatives thereof.

In the general processes, and schemes, that follow: R¹, R², R³, R⁴, R⁵, R⁶ and R⁷ are as previously defined unless otherwise stated; X is halo; Z is OH, or a carboxylic acid activating group such as chloro or 1H-imidazol-1-yl; Pg is an amino protecting group; BOC is tert-butoxycarbonyl; CBz is benzyloxycarbonyl; Bn is benzyl, Fmoc is 9-fluorenylmethoxycarbonyl; MeOH is methanol; EtOH is ethanol; EtOAc is ethyl acetate; Et₂O is diethyl ether; THF is tetrahydrofuran; DMSO is dimethyl sulfoxide; DCM is dichloromethane; AcOH is acetic acid; TFA is trifluoroacetic acid; STAB is sodium triacetoxyborohydride; DMA is N,N-dimethylacetamide; DMSO is dimethylsulphoxide; NMM is N-methylmorpholine; WSCDI is 1-(3-dimethylaminopropyl)-3-ethylcarbodiimide hydrochloride; DCC is N,N′-dicyclohexylcarbodiimide; HOBT is 1-hydroxybenzotriazole hydrate; PyBOP® is Benzotriazol-1-yloxytris(pyrrolidino)phosphonium hexafluorophosphate; PyBrOP® is bromo-tris-pyrrolidino-phosphonium; Hünig's base is N-ethyldiisopropylamine; Et₃N is triethylamine; HBTU is O-Benzotriazol-1-yl-N,N,N′,N′-tetramethyluronium hexafluorophosphate; L is a leaving group appropriate to aliphatic nucleophilic substitution, such as those, disclosed in Jerry March, ibid, page 352 (incorporated herein by reference), including Cl, Br, I and sulfonic esters (e.g. tosylate, mesylate and triflate).

According to a first process (A) compounds of formula (I) wherein R⁵ is COR⁶ may be prepared by reacting a compound of formula (XXIX)

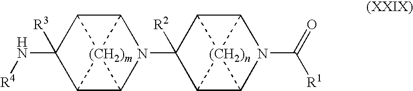

with a compound of formula (III)

R⁶COZ (III)

under conventional acid amine coupling conditions. Conveniently, the reaction may be effected as described in Scheme 1 step (g)

According to a second process (B) compounds of formula (I) wherein R⁵ is SO₂R⁷ may be prepared by reacting a compound of formula (XXIX)

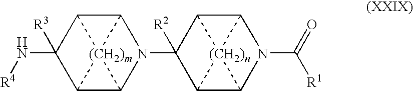

with a compound of formula (XXX)

R⁷SO₂X (XXX)

under conventional sulphonylation conditions. Conveniently, the reaction may be effected as described in Scheme 3 step (k)

According to a third process (C) compounds of formula (I) may be prepared by reacting a compound of formula (XXXI)

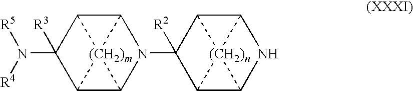

with a compound of formula (VII)

R¹COZ (VII)

under conventional acid amine coupling conditions. Conveniently, the reaction may be effected as described in Scheme 1a step (e)

According to a fourth process (D) compounds of formula (I) wherein R² is alkyl may be prepared by reacting a compound of formula (XXXII)

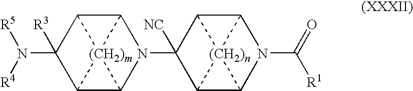

with a compound of formula (XI)

R²MgX (XI)

under conventional conditions. Conveniently, the reaction may be effected as described in Scheme 1c step (b)

According to a further process (E) compounds of formula (I) may be prepared from other compounds of formula (I) by functional group interconversion under conventional conditions.

Schemes that further illustrate general methods for the preparation of compounds of formula (I), and intermediates thereto, follow.

It will be appreciated by those skilled in the art that certain of the procedures described in the schemes for the preparation of compounds of formula (I) or intermediates thereto may not be applicable to some of the possible substituents.

It will be further appreciated by those skilled in the art that it may be necessary or desirable to carry out the transformations described in the schemes in a different order from that described, or to modify one or more of the transformations, to provide the desired compound of formula (I).

It will be still further appreciated by those skilled in the art that, as illustrated in the schemes that follow, it may be necessary or desirable at any stage in the synthesis of compounds of formula (I) to protect one or more sensitive groups in the molecule so as to prevent undesirable side reactions. In particular, it may be necessary or desirable to protect amino groups. The protecting groups used in the preparation of compounds of formula (I) may be used in conventional manner. See, for example, those described in ‘Protective Groups in Organic Synthesis’ by Theodora W Green and Peter G M Wuts, third edition, (John Wiley and Sons, 1999), in particular chapter 7, pages 494-653 (“Protection for the Amino Group”), incorporated herein by reference, which also describes methods for the removal of such groups.

The amino protecting groups t-butoxycarbonyl (Boc), 9-fluorenylmethoxycarbonyl (Fmoc), benzyloxycarbonyl (Cbz), methylformate, benzyl and acetyl are of particular use in the preparation of compounds of formula (I) and intermediates thereto.

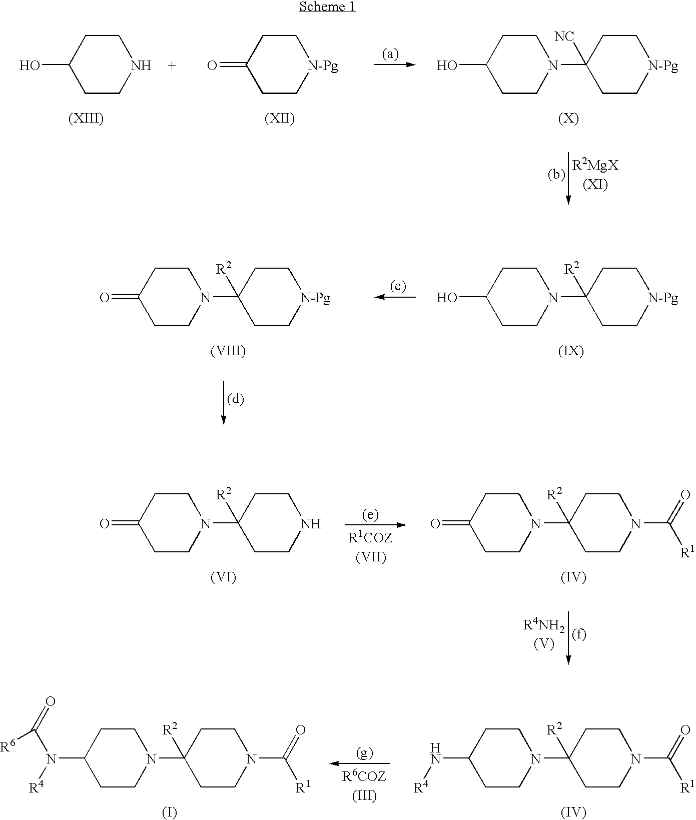

Scheme 1 illustrates the preparation of formula (I) wherein R³ is H and R⁵ is COR⁶.

With specific reference to scheme 1, the transformations depicted therein may be effected as follows:

Step (a): Compounds of formula (X) may be prepared by reacting compounds of formula (XIII), or O-protected analogues thereof, with a compound of formula (XII) in the presence of a suitable cyanating agent (e.g. Et₂AlCN (J. Am. Chem. Soc. 94 (13), 4635, 1972), acetone cyanohydrin, or an acid such as acetic acid, sulphuric acid, NaHSO₄, KHSO₃ or Na₂S₂O₅ and a cyanide source such as NaCN, KCN, trimethylsilylcyanide, glycolonitrile or dimethylaminoacetonitrile); optionally in the presence of Ti(ⁱOPr)₄; in a solvent such as a haloalkane (e.g. DCM or dichloroethane) or THF; at a temperature between 0° C. and 100° C. (e.g between 0° C. and 50° C., conveniently at ambient temperature)

Alternatively compounds of formula (X) may be generated by the action of HCN on the corresponding imine which may be either preformed or formed in situ from the reaction of a compound of formula (XIII) and a compound of formula (XII) in the presence of a solvent. If a compound of formula (XIII) is a protected derivative thereof, this may be removed subsequent to step (a) to provide a compound of formula (X) or step (b) to provide a compound of formula (IX).

Step (b): Compounds of formula (X) may be converted to compounds of formula (IX) via a Bruylants Reaction (e.g. C. Agami, F. Couty, G. Evano Organic Letters 2000, 14(2), 2085-2088). A compound of formula (IX) may be prepared by reacting a compound of formula (X) with an organometallic agent such as a Grignard Reagent of formula (XI), R²MgBr, or an organolithium reagent of formula R²Li; optionally in the presence of trimethylaluminium; in a solvent such as THF or Et₂O; at a temperature between 0° C. and ambient. Conveniently an excess of Grignard Reagent may be used.
Step (c) Ketones of formula (VIII) may be prepared by oxidation of alcohols of formula (IX) using methods well known in the literature (see for example Comprehensive Organic Synthesis Volume 8: Oxidation, Ed. B. M. Trost and I. Fleming, Pergamon Press 1991). One preferred method is the Swern Reaction.
Step (d) Deprotection of compounds of formula (VIII) may be undertaken using standard methodology. Preferred protecting groups include BOC whereupon deprotection may be effected using TFA or HCl in a solvent such as an ether (e.g. diethyl ether), a haloalkane (e.g. DCM) or ethyl acetate). Conveniently the reaction is performed at a temperature between 0° C. to RT. Alternative preferred protecting groups include Bn, CBz and Fmoc which may be deprotected by methods known to those skilled in the art.
Step (e) Compounds of formula (IV) may be prepared by reacting compounds of formula (VI) with compounds of formula (VII) under conventional acid amine coupling conditions. The acid amine coupling is conveniently effected using an amine of formula (IV) and an acid chloride of formula (VII); an excess of an acid acceptor, such as triethylamine or Hünig's base or an inorganic base such as potassium carbonate; in a solvent, such as a haloalkane (e.g. DCM); and at ambient temperature.

Alternatively, the acid/amine coupling is effected using an acid of formula (IV) activated by reagents such as WSCDI or DCC and HOBt or HOAt; an excess of an acid acceptor such as triethylamine or N-ethyl-N,N-diisopropylamine; in a solvent such as NMM or DCM; at ambient temperature. Alternatively, PYBOP®/PyBrOP® or Mukaiyama's reagent may be used under standard conditions.

Step (f) Compounds of formula (II) may be reacting compounds of formula (IV) with compounds of formula (V) under conventional reductive animation conditions. Conveniently, reductive amination may be effected by reacting compounds of formula (IV) with amines of formula (V), R⁴NH₂, in the presence of a reducing agent such as NaBH₄, Na(OAc)₃BH, NaCNBH₃; optionally in the presence of NaOAc or AcOH; optionally in the presence of an additive such as titanium tetraisopropoxide optionally in the presence of a drying agent such as MgSO₄ or molecular sieves; in a solvent such as DCM, methanol or DCE.
Step (g) Acid amine coupling may be effected according to the conditions described above in step (e).

In another variation of scheme 1, compounds of formula (I) may be prepared by carrying out steps (d) to (g) in a different order, such as scheme 1a wherein the order is (f), (g), (d), (e).

In further variations of scheme 1, compounds of formula (I) may be prepared by carrying out steps (a) to (g) in a different order, as illustrated in schemes 1b and 1c that follows:

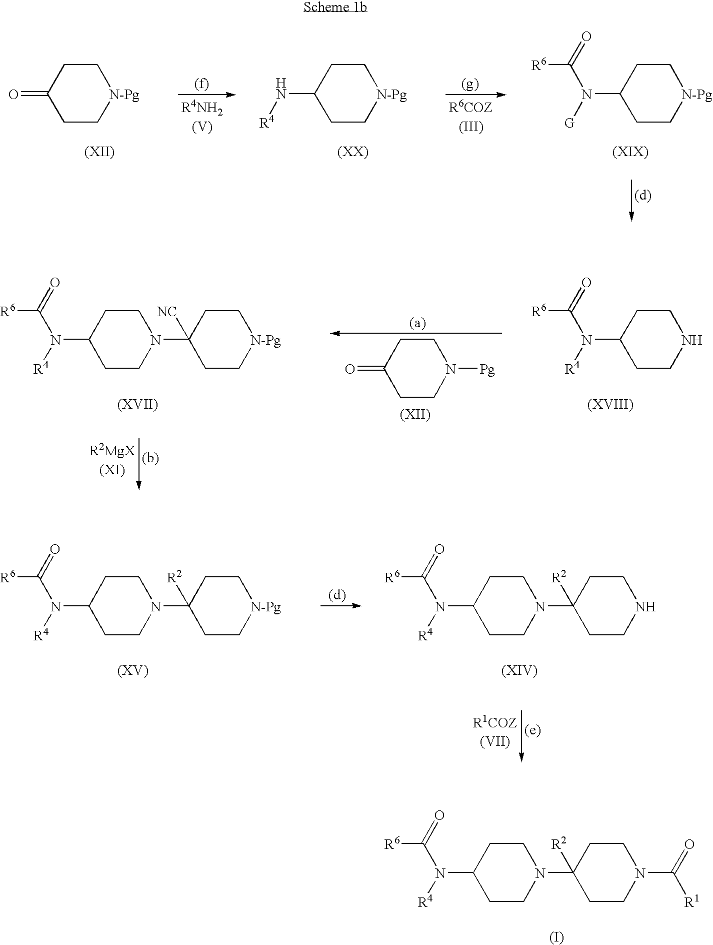

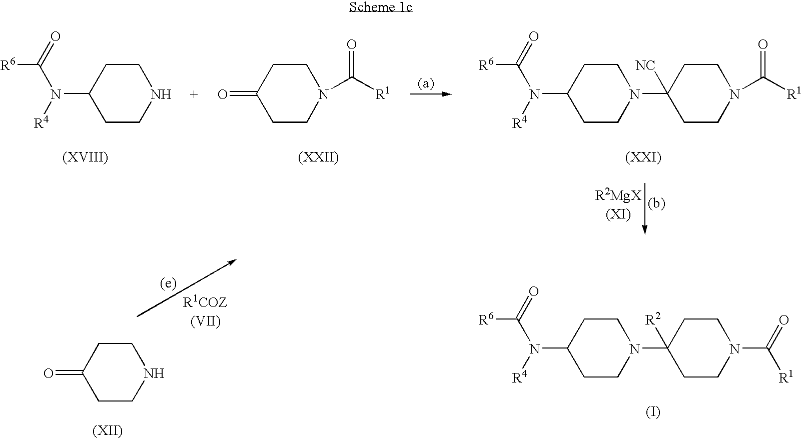

Compounds of formula (I) may also be prepared from a compound of formula (XXIII)

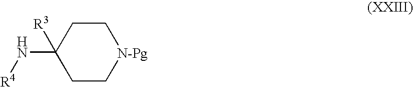

according to the transformations described in Schemes 1b and 1c for the preparation of compounds of formula (I) from compounds of formula (XX). Compounds of formula (XXIII) may be prepared according to Scheme 2a or 2b:

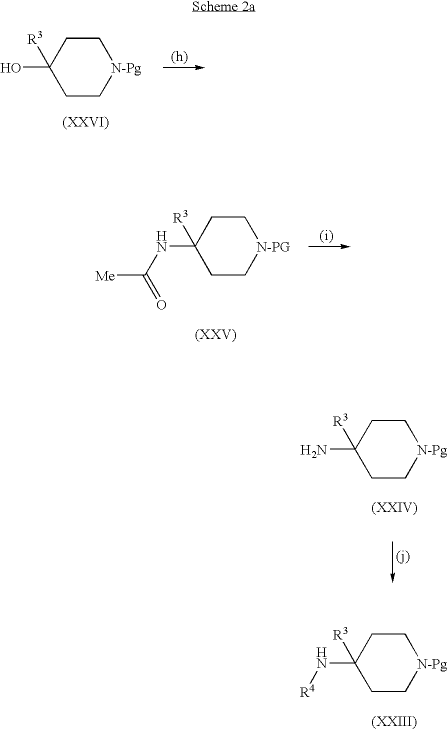

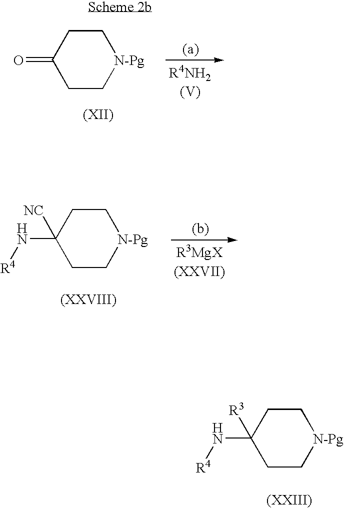

With specific reference to Scheme 2a, the transformations depicted therein may be effected as follows:

Step (h) Compounds of formula (XXV) may be prepared from compounds of formula (XXVI) under conventional conditions. Conveniently, compounds of formula (XXV) may be prepared from compounds of formula (XXVI) via the Ritter Reaction of a compound of formula (XXVI) with acetonitrile and a concentrated acid, such as sulphuric acid.
Step (i) Compounds of formula (XXIV) may be prepared by hydrolysis of acetamides of formula (XXV) under conventional conditions. Conveniently, hydrolysis may be effected in the presence of a strong mineral acid (such as HCl) at elevated temperatures.
Step (j) The primary amine of formula (XXIV) may be converted to the secondary, amine of formula (XXIII) through the use of standard conditions. Conveniently, compounds of formula (XXIII) may be prepared by reductive amination of a compound of formula (XXIV) with an aldehyde of formula R⁴C(O)H, according to the conditions described in Step (f).

Alternatively, compounds of formula (XXIII) may be prepared by [symbol] of compounds of formula (XXIV) using a compound of formula R⁴—L optionally in the presence of an base such as triethylamine, Hünigs base, or potassium carbonate.

A person skilled in the art will appreciate that compounds of formula (I) wherein R³ is alkyl may also be prepared according to Schemes 1, 1a, 1b and 1c when the reductive amination step (f) is replaced by transformations (a) and (b) described in Scheme 2b.

By analogy, a person skilled in the art will further appreciate that compounds of formula (I) wherein R² is hydrogen may be prepared according to Schemes 1, 1a, 1b and 1c when transformations (a) and (b) are replaced by reductive amination step (f).

Compounds of formula (I) wherein R⁵ is SO₂R⁷ may be prepared by methods which are directly analogous to preparation of compounds of formula (I) wherein R⁵ is COR⁶. In particular, compounds of formula (I) wherein R⁵ is SO₂R⁷ may be prepared according to Schemes 1, 1a, 1b and 1c when the acid amine coupling step (g) is replaced by standard sulphonylation conditions known to those skilled in the art. Sulphonation may conveniently be effected according to Scheme 3.

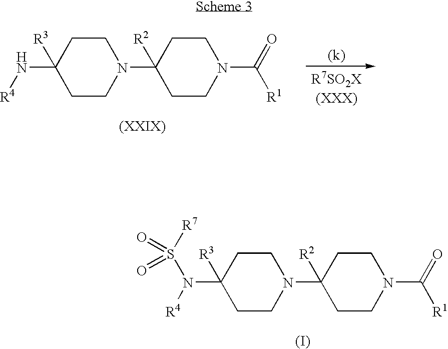

Step (k) Compounds of formula wherein R⁵ is SO₂R⁷ may be prepared by reacting compounds of formula (XXIX) with a sulphonylating agent such as a compound of formula (XXX), R⁷SO₂X, conveniently a sulphonyl chloride or sulphonyl fluoride.

A person skilled in the art will further appreciate that compounds of formula (I) wherein m or n≠0, i.e., which contain a bridged piperidine ring, may be prepared according to any of the above schemes using the corresponding bridged piperidine derivatives.

Compounds of formulae (III), (V), (VII), (XI), (XII), (XIII), (XXVII) and (XXX) are either known compounds or may be prepared by conventional chemistry

The compounds of formula (I) and their pharmaceutically acceptable salts, solvates and derivatives are useful because they have pharmacological activity in animals, including humans. More particularly, they are useful in the treatment of a disorder in which the modulation, in particular antagonism, of CCR5 receptors is implicated. Disease states of particular interest include HIV, retroviral infections genetically related to HIV, AIDS, inflammatory diseases, autoimmune diseases and pain.

The compounds of this invention may be used for treatment of respiratory disorders, including adult respiratory distress syndrome (ARDS), bronchitis, chronic bronchitis, chronic obstructive pulmonary disease, cystic fibrosis, asthma, emphysema, rhinitis, chronic sinusitis, sarcoidosis, farmer's lung, nasal polyposis, fibroid lung or idiopathic interstitial pneumonia.

Other conditions that may be treated are those triggered, affected or are in any other way correlated with T-cell trafficking in different organs. It is expected that the compounds of this invention may be useful for the treatment of such conditions and in particular, but not limited to, conditions for which a correlation with CCR5 or CCR5 chemokines has been established, and more particularly, but not limited to, the following: multiple sclerosis; Behcet's disease, Sjogren's syndrome or systemic sclerosis; arthritis, such as rheumatoid arthritis, spondyloarthropathies, gouty arthritis, osteoarthritis, systemic lupus erythematosus, and juvenile arthritis; and graft rejection, in particular, but not limited to, solid organ transplants, such as heart, lung, liver, kidney and pancreas transplants (e.g. kidney and lung allografts), and graft versus host rejection; inflammatory bowel disease, including Crohn's disease and ulcerative colitis; inflammatory lung conditions; endometriosis; renal diseases, such as glomerular disease (e.g. glomerulonephritis); fibrosis, such as liver, pulmonary and renal fibrosis; encephalitis, such as HIV encephalitis; chronic heart failure; myocardial infarction; hypertension; stroke; ischaemic heart disease; atherosclerotic plaque; restenosis; obesity; psoriasis; atopic dermatitis; CNS diseases, such as AIDS related dementias and Alzheimer's disease; anaemia; chronic pancreatitis; Hashimoto's thyroiditis; type I diabetes; cancer, such as non-Hodgkin's lymphoma, Kaposi's sarcoma, melanoma and breast cancer; pain, such as nociceptive pain and neuropathic pain (e.g. peripheral neuropathic pain); and stress response resulting from surgery, infection, injury or other traumatic insult.

Infectious diseases where modulation of the CCR5 receptor is implicated include acute and chronic hepatitis B Virus (HBV) and hepatitis C Virus (HCV) infection; bubonic, septicemic, and pneumonic plague; pox virus infection, such as smallpox; toxoplasmosis infection; mycobacterium infection; trypanosomal infection such as Chagas' Disease; pneumonia; and cytosporidiosis.

For a recent review of possible applications of chemokines and chemokine receptor blockers see Cascieri, M. A., and Springer, M. S., “The chemokine/chemokine receptor family: potential and progress for therapeutic intervention”, Curr. Opin. Chem. Biol., 4(4), 420-7 (August 2000).

Accordingly, in another aspect the invention provides a compound of formula (I) or a pharmaceutically acceptable salt, solvate or derivative thereof for use as a medicament.

In another aspect the invention provides a compound of formula (I) or a pharmaceutically acceptable salt, solvate or derivative thereof, for the treatment of a disorder in which the modulation of CCR5 receptors is implicated.

In another aspect the invention provides a compound of formula (I) or a pharmaceutically acceptable salt, solvate or derivative thereof, for the treatment of HIV, a retroviral infection genetically related to HIV, AIDS, an inflammatory disease, autoimmune disease and pain.

In another aspect the invention provides a compound of formula (I) or a pharmaceutically acceptable salt, solvate or derivative thereof, for the treatment of a respiratory disorder including adult respiratory distress syndrome (ARDS), bronchitis, chronic bronchitis, chronic obstructive pulmonary disease, cystic fibrosis, asthma, emphysema, rhinitis or chronic sinusitis, sarcoidosis, farmer's lung, nasal polyposis, fibroid lung or idiopathic interstitial pneumonia.

In another aspect the invention provides a compound of formula (I) or a pharmaceutically acceptable salt, solvate or derivative thereof, for the treatment of multiple sclerosis, Behcet's disease, Sjogren's syndrome, systemic sclerosis, rheumatoid arthritis or graft rejection.

In another aspect the invention provides a compound of formula (I) or a pharmaceutically acceptable salt, solvate or derivative thereof, for the treatment of inflammatory bowel disease; inflammatory lung conditions; endometriosis; renal diseases; fibrosis; encephalitis; chronic heart failure; myocardial infarction; hypertension; stroke; ischaemic heart disease; restenosis; atherosclerotic plaque; obesity; psoriasis; CNS diseases; anaemia; atopic dermatitis; chronic pancreatitis; Hashimoto's thyroiditis; type I diabetes; cancer; pain; or stress response resulting from surgery, infection, injury or other traumatic insult.

In another aspect the invention provides a compound of formula (I) or a pharmaceutically acceptable salt, solvate or derivative thereof, for the treatment of HBV, HCV, plague, pox virus, toxoplasmosis, mycobacterium, trypanosomal, pneumonia, or cytosporidiosis.

In another aspect the invention provides the use of a compound of formula (I) or of a pharmaceutically acceptable salt, solvate or derivative thereof, for the manufacture of a medicament for the treatment of a disorder in which the modulation of CCR5 receptors is implicated.

In another aspect the invention provides a method of treatment of a mammalian disorder in which the modulation of CCR5 receptors is implicated which comprises treating said mammal with an effective amount of a compound of formula (I) or with a pharmaceutically acceptable salt, solvate or derivative thereof.

The compounds of the invention may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or in any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Suitable modes of administration include oral, parenteral, topical, inhaled/intranasal, rectal/intravaginal, and ocular/aural administration.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 0.1 wt % to 80 wt %, more typically from 1 wt % to 60 wt %, such as 5 wt % to 50 wt %, of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 0.1 wt % to 25 wt %, more typically from 0.5 wt % to 20 wt %, such as 1 wt % to 15 wt %, of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch, calcium carbonate and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavours, preservatives and taste-masking agents.

Exemplary tablets contain up to about 80% drug, from about 10 wt % to about 90 wt % binder, from about 0 wt % to about 85 wt % diluent, from about 1 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the compound. Examples of such formulations include drug-coated stents and PGLA microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound comprising, for example, ethanol (optionally, aqueous ethanol) or a suitable alternative agent for dispersing, solubilising, or extending release of the compound, the propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable, solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid) (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of the invention. The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, vaginal ring or enema. Cocoa butter is a traditional suppository base, but various alternatives may be used as appropriate. As described hereinabove, the compounds of the invention can also be applied topically to mucosa, such as vaginal and rectal mucosa. Typical formulations for this purpose include gels, creams, ointments, foams, wafers, implants and sponges.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for, these purposes are alpha-, beta- and gamma-cyclodextrins, examples of which may be found in International Patent Applications Nos. WO 91/11172, WO 94/02518 and WO 98/55148.

Inasmuch as it may desirable to administer a compound of the invention in combination with another therapeutic agent, for example, for the purpose of treating a particular disease or condition, it is within the scope of the present invention that two or more pharmaceutical compositions, at least one of which contains a compound of the invention, may conveniently be combined in the form of a kit suitable for coadministration of the compositions.

Thus the kit of the invention comprises two or more separate pharmaceutical compositions, at least one of which contains a compound of formula (I) or a pharmaceutically acceptable salt, solvate or derivative thereof, and means for separately retaining said compositions, s [symbol] ontainer, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, having a weight of about 65 to 70 kg, the total daily dose of a compound of the invention is typically in the range 1 to 10,000 mg, such as 10 to 1,000 mg, for example 25 to 500 mg, depending, of course, on the mode of administration, the age, condition and weight of the patient, and will in any case be at the ultimate discretion of the physician. The total daily dose may be administered in single or divided doses.

Accordingly in another aspect the invention provides a pharmaceutical composition including a compound of formula (I) or a pharmaceutically acceptable salt, solvate or derivative thereof together with one or more pharmaceutically acceptable excipients, diluents or carriers.

The compounds of formula (I) and their pharmaceutically acceptable salts, solvates and derivatives have the advantage that they are more selective, have a more rapid onset of action, are more potent, are better absorbed, are more stable, are more resistant to metabolism, have a reduced ‘food effect’, have an improved safety profile or have other more desirable properties (e.g. with respect to solubility or hygroscopicity) than the compounds of the prior art.

The compounds of formula (I) and their pharmaceutically acceptable salts, solvates and derivatives may be administered alone or as part of a combination therapy. Thus included within the scope of the present invention are embodiments comprising co-administration of, and compositions which contain, in addition to a compound of the invention, one or more additional therapeutic agents.

Such multiple drug regimens, often referred to as combination therapy, may be used in the treatment and prevention of any of the diseases or conditions mediated by or associated with CCR5 chemokine receptor modulation, particularly infection by human immunodeficiency virus, HIV. The use of such combination therapy is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature. A recommended treatment for HIV is a combination drug treatment called Highly Active Anti-Retroviral Therapy, or HAART. HAART combines three or more HIV drugs. Thus, the methods of treatment and pharmaceutical compositions of the present invention may employ a compound of the invention in the form of monotherapy, but said methods and compositions may also be used in the form of combination therapy in which one or more compounds of the invention are co-administered in combination with one or more additional therapeutic agents such as those described in detail further herein.

The therapeutic agents that may be used in combination with the compounds of the present invention include, but are not limited to, those useful as HIV protease inhibitors (PIs), non-nucleoside reverse transcriptase inhibitors (NNRTIs), nucleoside/nucleotide reverse transcriptase inhibitors (NRTIs), CCR5 antagonists, agents which inhibit the interaction of gp120 with CD4, other agents which inhibit the entry of HIV into a target cell (such as fusion inhibitors), inhibitors of HIV integrase, RNaseH inhibitors, prenylation inhibitors, maturation inhibitors which act by interfering with production of the HIV capsid protein, compounds useful as anti-infectives, and others as described below.

It will be appreciated by a person skilled in the art, that a combination drug treatment, as described herein above, may comprise two or more compounds having the same, or different, mechanism of action. Thus, by way of illustration only, a combination may comprise a compound of the invention and: one or more NRTIs; one or more NRTIs and a PI; one or more NRTIs and another CCR5 antagonist; a PI; a PI and an NNRTI; an NNRTI; and so on.

Examples of PIs include, but are not limited to, amprenavir (141W94), CGP-73547, CGP-61755, DMP-450 (mozenavir), nelfinavir, ritonavir, saquinavir (invirase), lopinavir, TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, becanavir (formerly known as VX-478, GW640385), indinavir, tipranavir, TMC-114, DPC-681, DPC-684, fosamprenavir calcium (Lexiva), benzenesulfonamide derivatives disclosed in WO 03/053435, R-944, Ro-03-34649, VX-385, GS-224338, OPT-TL3, PL-100, PPL-100, SM-309515, AG-148, PG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005.

Examples of NRTIs include, but are not limited to, abacavir, GS-840, lamivudine, adefovir dipivoxil, beta-fluoro-ddA, zalcitabine, didanosine, stavudine, zidovudine, tenofovir disoproxil fumarate, amdoxovir (DAPD), SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine (FTC).

Examples of NNRTIs include, but are not limited to, efavirenz, HBY-097, nevirapine, TMC-120 (dapivirine), TMC-125, etravirine, delavirdine, DPC-083, DPC-961, capravirine, rilpivirine, 5-{[3,5-Diethyl-1-(2-hydroxyethyl)-1H-pyrazol-4-yl]oxy}isophthalonitrile or pharmaceutically acceptable salts, solvates or derivatives thereof; GW-678248, GW-695634, MIV-150, calanolide, and tricyclic pyrimidinone derivatives as disclosed in WO 03/062238.

Examples of CCR5 antagonists include, but are not limited to, TAK-779, SC-351125, ancriviroc (also known as SCH-C), vicriviroc (formerly known as SCH-D), maraviroc, PRO-140, aplaviroc (also known as GW-873140, Ono-4128, AK-602), AMD-887 CMPD-167, methyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or pharmaceutically acceptable salts, solvates or derivatives thereof, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate or pharmaceutically acceptable salts, solvates or derivatives thereof, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or pharmaceutically acceptable salts, solvates or derivatives thereof, and N-{(1S)-3-[3-endo-(5-Isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) or pharmaceutically acceptable salts, solvates or derivatives thereof.

Examples of entry and fusion inhibitors include, but are not limited to, BMS-806, BMS-488043, 5-{(1S)-2-[(2R)-4-Benzoyl-2-methyl-piperazin-1-yl]-1-methyl-2-oxo-ethoxy}-4-methoxy-pyridine-2-carboxylic acid methylamide and 4-{(1S)-2-[(2R)-4-Benzoyl-2-methyl-piperazin-1-yl]-1-methyl-2-oxo-ethoxy}-3-methoxy-N-methyl-benzamide, enfuvirtide (T-20), SP-01A, T1249, PRO542, AMD-3100, soluble CD4, compounds disclosed in JP 2003171381, and compounds disclosed in JP 2003119137.

Examples of inhibitors of HIV integrase include, but are not limited to, L-000870810 GW-810781, 1,5-naphthyridine-3-carboxamide derivatives disclosed in WO 03/062204, compounds disclosed in WO 03/047564, compounds disclosed in WO 03/049690, and 5-hydroxypyrimidine-4-carboxamide derivatives disclosed in WO 03/035076, MK-0518 (5-(1,1-dioxo-1,2-thiazinan-2-yl)-N-(4-fluorobenzyl)-8-hydroxy-1,6-naphthyridine-7-carboxamide—disclosed in WO 03016315).

Examples of prenylation inhibitors include, but are not limited to, HMG CoA reductase inhibitors, such as statins (e.g. atorvastatin).

Examples of maturation inhibitors include 3-O-(3′3′-dimethylsuccinyl) betulic acid (otherwise known as PA-457) and alphaHGA.

Anti-infectives that may be used in combination with the compounds of the present invention include antibacterials and antifungals. Examples of antibacterials include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R 51211, trimetrexate, daunorubicin, recombinant human erythropoietin, recombinant human growth hormone, megestrol acetate, testerone, and total enteral nutrition. Examples of antifungals include, but are not limited to, anidulafungin, C31G, caspofungin, DB-289, fluconazaole, itraconazole, ketoconazole, micafungin, posaconazole, and voriconazole.

There is also included within the scope the present invention, combinations of a compound of formula (I), or a pharmaceutically acceptable salt, solvate or derivative thereof, together with one or more additional therapeutic agents independently selected from the group consisting of:
Proliferation inhibitors, e.g. hydroxyurea.
Immunomodulators, such as AD-439, AD-519, alpha interferon, AS-101, bropirimine, acemannan, CL246.738, EL10, FP-21399, gamma interferon, granulocyte macrophage colony stimulating factor (e.g. sargramostim), IL-2, immune globulin intravenous, IMREG-1, IMREG-2, imuthiol diethyl dithio carbamate, alpha-2 interferon, methionine-enkephalin, MTP-PE, remune, rCD4, recombinant soluble human CD4, interferon alfa-2, SK&F106528, soluble T4 thymopentin, tumor necrosis factor (TNF), tucaresol, recombinant human interferon beta, interferon alfa n-3.
Tachykinin receptor modulators (e.g. NK1 antagonists) and various forms of interferon or interferon derivatives.
Other chemokine receptor agonists/antagonists such as CXCR4 antagonists (e.g AMD070 and AMD3100) or CD4 antagonists (e.g. TNX-355).
Agents which substantially inhibit, disrupt or decrease viral transcription or RNA replication such as inhibitors of tat (transcriptional trans activator) or nef (negative regulatory factor).
Agents which substantially inhibit, disrupt or decrease translation of one or more proteins expressed by the virus (including, but not limited to, down regulation of protein expression or antagonism of one or more proteins) other than reverse transcriptase, such as Tat or Nef.
Agents which influence, in particular down regulate, CCR5 receptor expression; chemokines that induce CCR5 receptor internalisation such MIP-1α, MIP-1β, RANTES and derivatives thereof; examples of such agents include, but are not limited to, immunosupressants, such as calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-{(3R,4R)-4-methyl-3-[methyl-(7H-pyrrolo[2,3-d]pyrimidin-4-yl)-amino]-piperidin-1-yl}-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab);
Agents which interfere with cell activation or cell cycling, such as rapamycin.

In addition to the requirement of therapeutic efficacy, which may necessitate the use of therapeutic agents in addition to the compounds of the invention, there may be additional rationales which compel or highly recommend the use of a combination of a compound of the invention and another therapeutic agent, such as in the treatment of diseases or conditions which directly result from or indirectly accompany the basic or underlying CCR5 chemokine receptor modulated disease or condition. For example, where the basic CCR5 chemokine receptor modulated disease or condition is HIV infection and multiplication it may be necessary or at least desirable to treat Hepatitis C Virus (HCV), Hepatitis B Virus (HBV), Human Papillomavirus (HPV), neoplasms, and other conditions which occur as the result of the immune-compromised state of the patient being treated. Other therapeutic agents may be used with the compounds of the invention, e.g., in order to provide immune stimulation or to treat pain and inflammation which accompany the initial and fundamental HIV infection.

Accordingly, therapeutic agents for use in combination with the compounds of formula (I) and their pharmaceutically acceptable salts, solvates and derivatives also include:
Agents useful in the treatment of hepatitis, such as interferons, pegylated interferons (e.g. peginterferon alfa-2a and peginterferon alfa-2b), long-acting interferons (e.g. albumin-interferon alfa); TLR7 inhibitors; reverse transcriptase inhibitors, such as lamivudine and emtricitabine; IMP dehydrogenase inhibitors such as ribavirin and viramidine; polymerase inhibitors (including NS5B polymerase inhibitors) such as valopicitabine, HCV-086, HCV-796 purine nucleoside analogues as disclosed in WO 05/009418, and imidazole derivatives as disclosed in WO 05/012288; alpha glucosidase inhibitors such as celgosivir; interferon enhancers such as EMZ-702; serine protease inhibitors such as B1LN-2061, SCH-6, VX-950, aza-peptide-based macrocyclic derivatives as disclosed in WO 05/010029 and those disclosed in WO 05/007681; caspase inhibitors such as IDN-6566; HCV replicon inhibitors such as arylthiourea derivatives as disclosed in WO 05/007601.
Agents useful in the treatment of AIDS related Kaposi's sarcoma, such as interferons, daunorubicin, doxorubicin, paclitaxel, metallo-matrix proteases, A-007, bevacizumab, BMS-275291, halofuginone, interleukin-12, rituximab, porfimer sodium, rebimastat, COL-3.
Agents useful in the treatment of cytomegalovirus (CMV), such as fomivirsen, oxetanocin G, cidofovir, cytomegalovirus immune globin, foscarnet sodium, Isis 2922, valacyclovir, valganciclovir, ganciclovir.
Agents useful in the treatment of herpes simplex virus (HSV), such as acyclovir, penciclovir, famciclovir, ME-609.

Further combinations for use according to the invention include combination of a compound of formula (I), or a pharmaceutically acceptable salt, solvate or derivative thereof with a CCR1 antagonist, such as BX-471; a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab).

There is also included within the scope the present invention, combinations of a compound of formula (I), or a pharmaceutically acceptable salt, solvate or derivative thereof, together with one or more additional therapeutic agents which slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir, saquinavir or ketoconazole.

In the above-described combinations, the compound of formula (I) or a pharmaceutically acceptable salt, solvate or derivative thereof and other therapeutic agent(s) may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Accordingly, in a further aspect the invention provides a pharmaceutical composition comprising a compound of formula (I) or a pharmaceutically acceptable salt, solvate or derivative thereof and one or more additional therapeutic agents.

It is to be appreciated that all references herein to treatment include curative, palliative and prophylactic treatment.

The invention is illustrated by the following Examples and Preparations in which the following further abbreviations may be used:

h=hour
min=minute
RT means room temperature
LRMS=low resolution mass spectrum
HRMS=high resolution mass spectrum
APCI=atmospheric pressure chemical ionisation
ESI=electrospray ionisation
NMR=nuclear magnetic resonance
HPLC means high-pressure liquid chromatography
tlc—thin layer chromatography
Me=methyl

### EXAMPLE 1

### N-benzyl-N-{1′-[(2,4-dimethylpyridin-3-yl)carbonyl]-4′-methyl-1,4′-bipiperidinyl-4-yl}-3,3-difluorocyclobutanecarboxamide

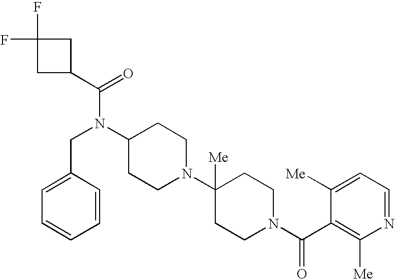

To a solution of preparation 10 (100 mg, 0.24 mmol) in dichloromethane (5 ml) was added difluorocyclobutanecarboxylic acid (50 mg, 0.37 mmol), triethylamine (100 ul, 0.72 mmol), 1-hydroxybenzotriazole hydrate (50 mg, 0.32 mmol) and 1-(3-dimethylaminopropyl)-3-ethylcarbodiimide hydrochloride (71 mg, 0.36 mmol), and the reaction mixture stirred at RT for 24 h. Saturated sodium bicarbonate (aq, 5 ml) was added, and the aqueous phase separated, extracted with dichloromethane (10 ml). The combined organics were dried over magnesium sulphate and dried in vacuo. Purification by column chromatography (silica, eluting with 95/5/0.5 dichloromethane/methanol/0.88 ammonia solution) gave the title compound as a colourless oil. The free base was converted to the dihydrochloride salt by the addition of HCl (1M in ether, 2 mL), and evaporated to dryness to afford a white solid (56 mg, 0.09 mmol, 36%).

¹H NMR (400 MHz CD₃OD) δ 0.93-1.04 (3H, m), 1.32-1.47 (1H, m), 1.46-1.83 (6H, m), 1.85-1.99 (1H, m), 2.12-2.31 (5H, m), 2.36-2.56 (4H, m), 2.66-3.12 (7H, m), 3.26-3.37 & 3.38-3.51 (1H, 2×m), 3.58-3.77 & 4.30-4.44 (2H, 2×m), 3.84-3.98 (1H, m), 4.54-4.66 (2H, s), 7.14-7.31 (5H, m), 7.32-7.41 (1H, m), 8.24-8.34 (1H, m).

LRMS: m/z APCI+540 [MH⁺].

Elemental analysis observed 57.45 (C %), 7.15 (H %), 8.56 (N %) calculated for C₃₁H₄₀F₂N₄O₂.2HCl.2H₂O 57.49 (C %), 7.16 (H %), 8.65 (N %) total mw=647.6

### EXAMPLES 2-7

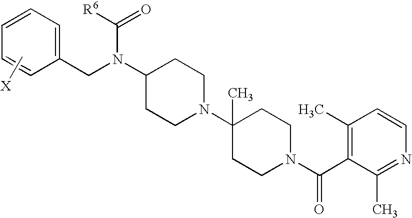

Examples 2 to 7 were prepared according to the method described above in Example 1 using the corresponding amine (preparations 10, 11 or 12) and the corresponding acid (R⁶CO₂H).

| Example | | | LRMS APCI |
|---|---|---|---|
| no. | R⁶ | X | [MH⁺]Data |
| 2 | 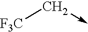 | H | 531 |
| 3 | 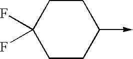 | H | 567 |
| 4 | 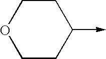 | H | 533 |
| 5^{A,B} | 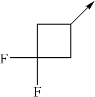 | 2-F | 557 |
| 6^{A} | 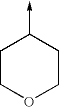 | 2-F | 551 |
| 7^{A} | 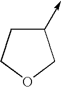 | 2-F | 537 |

^{A}= 1.5 eq. of acid was used and the reaction was stirred for 48 hours.
^{B}= isolated as a free base.

### EXAMPLE 5

### NMR

¹H NMR (400 MHz CD₃OD) δ 0.88-0.95 (3H, m), 1.18-1.78 (9H, m), 1.86-1.99 (1H, m), 2.05-2.29 (5H, m), 2.38-2.59 (4H, m), 2.68-3.10 (6H, m), 3.12-3.33 (1H, m), 3.34-3.57 (1H, m), 4.04-4.21 (1H, m), 4.42-4.53 & 4.61-4.66 (2H, 2×m), 6.94-7.34 (5H, m), 8.31-8.37 (1H, m).

### EXAMPLE 8

### N-benzyl-N-[1′-(3,5-dichloroisonicotinoyl)-4′-methyl-1,4′-bipiperidin-4-yl]-3,3-difluorocyclobutanecarboxamide.2HCl

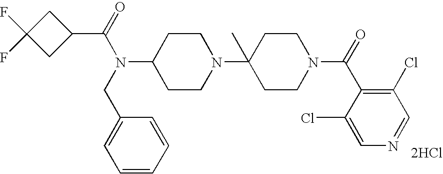

3,5-Dichloroisonicotinic acid (84 mg, 0.4 mmol), the compound of Preparation 42 (150 mg, 0.3 mmol), 3-(diethoxyphosphoryloxy)-1,2,3-benzotriazin-4(3H)-one (132 mg, 0.4 mmol) and triethylamine (0.16 mL, 1.2 mmol) were dissolved in dichloromethane and stirred at room temperature for 24 hours. The reaction was quenched by the addition of saturated sodium hydrogen carbonate solution and extracted using dichloromethane. The combined organic extracts were concentrated in vacuo to give the crude product. The crude mixture was purified by column chromatography on silica gel using dichloromethane:methanol (100:0 to 90:10) as eluent. The resulting product was then dissolved in dichloromethane (5 mL) and treated with 2M hydrochloric acid in diethyl ether (5 mL), the solvents were removed in vacuo to give 43 mg of title compound as a white solid.

¹H NMR (400 MHz CDCl₃) δ 0.95 (3H, s), 1.20-1.85 (9H, m), 1.85-2.00 (1H, m), 2.05-2.30 (2H, m), 2.45 (1H, bs), 2.70-3.10 (6H, m), 3.25-3.50 (2H, m), 4.05-4.20 (1H, m), 4.40-4.65 (2H, m), 7.10-7.40 (5H, m), 8.50 (2H, bs).

LRMS: m/z APCI+579[MH⁺]

### EXAMPLE 9

### N-benzyl-N-[1′-(3,5-dichloro-1-oxidoisonicotinoyl)-4′-methyl-1,4′-bipiperidin-4-yl]-3,3-difluorocyclobutanecarboxamide.HCl

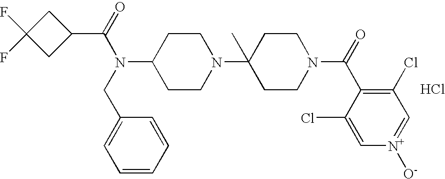

The title compound was prepared according to the method of Example 8 using 3,5-dichloro-1-oxy-isonicotinic acid (92 mg, 0.4 mmol) and the compound of Preparation 42 to give 38 mg of title compound as a white solid.

¹H NMR (400 MHz CDCl₃) δ 0.90 (3H, s), 1.25-1.95 (9H, m), 2.00-2.20 (2H, m), 2.40 (1H, bs), 2.65-3.00 (6H, m), 3.25-3.50 (2H, m), 3.95-4.10 (1H, m), 4.35-4.60 (2H, m), 7.05-7.35 (5H, m), 8.10 (2H, bs).

LRMS: m/z APCI+595[MH⁺]

### EXAMPLE 10

### N-benzyl-N-(1-{(8-syn)-3-[(4,6-dimethylpyrimidin-5-yl)carbonyl]-3-azabicyclo}piperidin-4-yl)cyclopropanecarboxamide

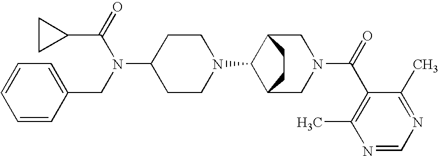

To a solution of the compound of preparation 36 (145 mg, 0.39 mmol) in dichloromethane (10 mL) were added 4,6-dimethylpyrimidine-5 carboxylic acid (U.S. Pat. No. 6,391,865 B1, p. 45) (72 mg, 0.5 mmol), 1-(3-dimethylaminopropyl)-3-ethylcarbodiimide hydrochloride (113 mg, 0.6 mmol), 1-hydroxybenzotriazole hydrate (91 mg, 0.6 mmol) and triethylamine (220 μL, 1.6 mmol). The reaction mixture was stirred at room temperature for 24 hours. The mixture was diluted with adding water (10 mL) and extracted with dichloromethane (3×20 mL). The combined organic extracts were washed with brine (10 mL), dried over magnesium sulfate and reduced in vacuo to give the crude residue. Purification by column chromatography on silica gel using dichloromethane:methanol:0.88 ammonia (97.5:2.5:0.25) gave 165 mg (83%) of the title compound as a white solid.

¹H NMR (400 MHz, CDCl₃) δ 0.66-0.86 (2H, m), 1.00-1.11 (2H, m), 1.34-1.55 (2H, m), 1.60-1.91 (6H, m), 1.98-2.09 (2H, m), 2.20 (1H, m), 2.31-2.45 (3H, m), 2.50-2.52 (3H, m), 2.61-2.70 (2H, m), 2.77-3.14 (3H, m), 3.29 (1H, d), 3.47 (1H, d), 3.65-3.78 (1H, m), 3.98-4.18 (1H, dd), 4.49-4.61 (1H, m), 4.71 (1H, s), 4.90 (1H, s), 7.17-7.39 (5H, m), 8.94 (1H, s).

LRMS: m/z APCI+502 [MH⁺].

### EXAMPLE 11

### N-benzyl-N-((8-syn)-3-{1-[(4,6-dimethylpyrimidin-5-yl)carbonyl]piperidin-4-yl}-3-azabicyclo[3.2.1]oct-8-yl)cyclopropanecarboxamide

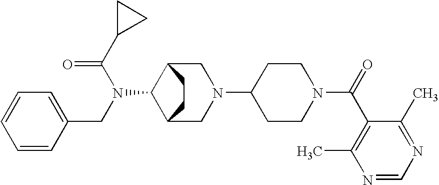

The title compound was prepared according to the method of Example 10 using the compound of preparation 39 (26 mg, 0.07 mmol) and 4,6-dimethylpyrimidine-5 carboxylic acid (U.S. Pat. No. 6,391,865 B1, p. 45) to give the title compound as a colourless gum (23 mg, 66%).

¹H NMR (400 MHz, CD₃OD) δ 0.82-0.87 (2H, m), 0.94-0.97 (2H, m), 1.74-1.88 (6H, m), 2.11-2.17 (2H, m), 2.32-2.35 (1H, m), 2.54 (3H, s), 2.65 (3H, s), 2.87-2.94 (1H, m), 3.08-3.25 (4H, m), 3.32-3.44 (3H, m), 3.56-3.60 (1H, m), 4.85-4.88 (1H, m), 4.99 (2H, s), 7.29-7.33 (5H, m), 7.38-7.42 (2H, m), 9.17 (1H, s).

LRMS: m/z APCI+502 [MH⁺].

### EXAMPLE 12

### N-benzyl-N-((8-syn)-3-{1-[(2,4-dimethylpyridin-3-yl)carbonyl]piperidin-4-yl}-3-azabicyclo[3.2.1]oct-8-yl)cyclopropanecarboxamide

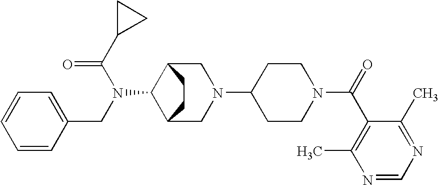

The title compound was prepared from the compound of preparation 39 (45 mg, 0.1 mmol) and 2,4-dimethyl-3-carboxypyridine (J. Am. Chem. Soc. 101 (23), 7036, 1979) (28 mg, 0.2 mmol) according to the method described above in Example 10, as a white solid in 77% yield.

¹H NMR (400 MHz, CD₃OD) δ 0.70-0.86 (2H, m), 0.98-1.07 (2H, m), 1.50-1.64 (6H, m), 1.78-2.01 (3H, m), 2.08-2.12 (1H, m), 2.18-2.21 & 2.50-2.53 (3H, 2×m), 2.24-2.31 (1H, m), 2.36-2.40 (3H, m), 2.43-2.46 (1H, m), 2.58-3.07 (5H, m), 3.14-3.49 (3H, m), 4.81-4.88 (2H, m), 5.00-5.03 (1H, m), 6.98-7.04 (1H, m), 7.15-7.22 (2H, m), 7.28-7.40 (3H, m), 8.34-8.37 (1H, m).

LRMS: m/z APCI+501 [MH⁺].

### EXAMPLE 13

### N-benzyl-N-{1′-[(2,4-dimethylpyridin-3-yl)carbonyl]-4′-methyl-1,4′-bipiperidin-4-yl}-N²,N²-dimethylglycinamide

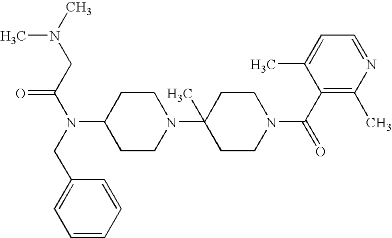

N,N-Dimethylglycine (0.37 mg, 0.4 mmol), the compound of preparation 10 (100 mg, 0.2 mmol), 1-(3-dimethylaminopropyl)-3-ethylcarbodiimide hydrochloride (71 mg, 0.4 mmol), 1-hydroxybenzotriazole hydrate (50 mg, 0.4 mmol) and triethylamine (100 μL, 0.24 mmol) in dichloromethane (5 mL) were stirred at room temperature for 48 hours. A solution of sodium hydrogen carbonate was then added to the reaction mixture, the phases separated and the aqueous layer washed with dichloromethane (2×10 mL). The organic extracts were then combined, dried over magnesium sulfate and concentrated in vacuo. Purification by column chromatography on silica gel using dichloromethane:methanol (90:5) as eluent afforded the title compound as a solid, 18.5 mg (15%).

¹H NMR (400 MHz, CD₃OD) δ 0.95-1.02 (3H, m), 1.32-1.46 (1H, m), 1.47-1.58 (1H, m), 6.32 (5H, m), 1.86-1.97 (1H, m), 2.13-2.31 (9H m), 2.35 (3H, s), 2.38-2.46 (3H, m), 2.87-2.97 (1H, m), 2.99-3.08 (2H, m), 3.08-3.14 (1H, m), 3.34-3.39 (1H, m), 3.60-3.74 (1H, m), 3.85-3.98 & 4.30-4.44 (2H, 2×m), 4.58-4.71 (2H, m), 7.14-7.30 (5H, m), 7.31-7.39 (1H, m), 8.27-8.33 (1H, m).

LRMS: m/z APCI+506 [MH⁺].

### EXAMPLE 14

### N-benzyl-N-{1′-[(2,4-dimethylpyridin-3-yl)carbonyl]-4′-methyl-1,4′-bipiperidin-4-yl}-2-ethoxyacetamide

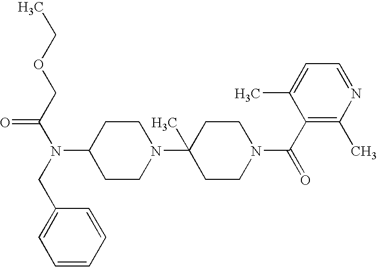

The title compound was prepared from ethoxyacetic acid (37 mg, 0.4 mmol) and the compound of preparation 10 (100 mg, 0.2 mmol) according to the method described above in Example 13 in 16% yield.

¹H NMR (400 MHz, CD₃OD) δ 0.95-1.03 (3H, m), 1.11-1.17 & 1.21-1.28 (3H, 2×m), 1.33-1.46 (7H, m), 1.47-1.59 (1H, m), 2.11-2.30 (5H, m), 2.38-2.46 (3H, m), 2.87-2.96 (1H, m), 2.98-3.09 (2H, m), 3.27-3.37 (1H, m), 3.43-3.52 (1H, m), 3.57-3.71 (2H, m), 3.72-3.82 & 4.28-4.38 (2H, 2×m), 3.86-3.96 (1H, m), 4.08-4.09 (1H, m), 4.55-4.64 (2H, m), 7.14-7.30 (5H, m), 7.31-7.39 (1H, m), 8.27-8.32 (1H, m).

LRMS: m/z APCI+507 [MH⁺].

### EXAMPLE 15

### N-benzyl-N-{1′-[(2,4-dimethylpyridin-3-yl)carbonyl]-4′-methyl-1,4′-bipiperidin-4-yl}-2-methoxyacetamide

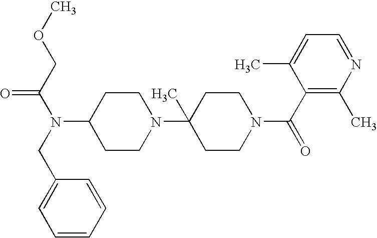

The title compound was prepared from methoxyacetic acid (32 mg, 0.4 mmol), and the compound of preparation 10 (100 mg, 0.2 mmol) according to the method described above in Example 13 in 40% yield as a solid.

¹H NMR (400 MHz, CD₃OD) δ 0.95-1.01 (3H, m), 1.32-1.45 (1H, m), 1.47-1.59 (1H, m), 1.61-1.81 (5H, m), 1.86-1.97 (1H, m), 2.10-2.30 (5H, m), 2.38-2.45 (3H, m), 2.86-2.95 (1H, m), 2.98-3.09 (2H, m), 3.26-3.37 & 3.42-3.47 (4H, 2×m), 3.59-3.75 & 4.28-4.41 (3H, 2×m), 3.86-3.97 (1H, m), 4.01-4.06 (1H, m), 4.54-4.64 (2H, m), 7.14-7.31 (5H, m), 7.32-7.40 (1H, m), 8.27-8.32 (1H, m).

LRMS: m/z APCI+493 [MH⁺].

### EXAMPLE 16

### N-benzyl-N-{1′-[(2,4-dimethylpyridin-3-yl)carbonyl]-4′-ethyl-1,4′-bipiperidin-4-yl}cyclopropanecarboxamide

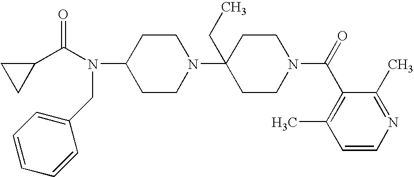

N-Ethyldiisopropylamine (1.3 mL, 7.3 mmol) was added to a stirred solution of the compound of preparation 23 (0.8 g, 2.1 mmol), 2,4-dimethyl-3-carboxypyridine (J. Am. Chem. Soc. 101 (23), 7036, 1979) (0.4 g, 2.1 mmol), 1-(3-dimethylaminopropyl)-3-ethylcarbodiimide hydrochloride (0.5 g, 2.5 mmol) and 1-hydroxybenzotriazole hydrate (0.3 g, 2.5 mmol) in dichloromethane (10 mL). The reaction mixture was stirred for 48 hours and diluted with saturated sodium hydrogen carbonate solution. The aqueous layer was extracted with ethyl acetate (3×50 mL). The combined organic extracts were washed with brine, dried over magnesium sulfate and the solvent removed in vacuo. Purification by column chromatography on silica gel using dichloromethane:methanol:0.88 ammonia (95:5:0.5-90:10:1) as eluent gave 0.5 g (47%) of the title compound as a white solid.

¹H NMR (400 MHz, CD₃OD) δ 0.66-0.98 (6H, m), 1.33-2.12 (12H, m), 2.23-2.45 (8H, m), 2.88-3.09 (3H, m), 3.25-3.39 (2H, m), 4.14-4.44 (2H, m), 4.61-4.67 & 4.81 (2H, 2×m), 7.15-7.39 (6H, m), 8.30 (1H, d).

LRMS: m/z APCI+503 [MH⁺].

### EXAMPLE 17

### N-benzyl-N-{1′-[(2,4-dimethylpyridin-3-yl)carbonyl]-4′-isopropyl-1,4′-bipiperidin-4-yl}cyclopropanecarboxamide

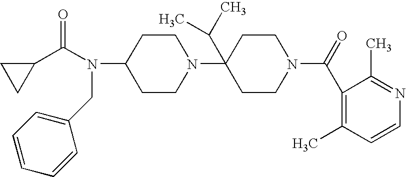

The title compound was prepared according to the method of Example 16 using the compound of preparation 22 (0.4 g, 1.1 mmol) and 2,4-dimethyl-3-carboxypyridine to give the title compound as a white solid (0.4 g, 67%).

¹H NMR (400 MHz, CD₃OD) δ 0.66-0.99 (10H, m), 1.29-2.13 (10H, m), 2.22-2.57 (8H, m), 2.97-3.35 (5H, m), 4.17-4.83 (4H, m), 7.16-7.39 (6H, m), 8.30 (1H, m).

LRMS: m/z APCI+517 [MH⁺].

### EXAMPLE 18

### N-{1′-[(2,4-dimethylpyridin-3-yl)carbonyl]-4′-methyl-1,4′-bipiperidin-4-yl}-3,3-difluoro-N-(3-fluorobenzyl)cyclobutanecarboxamide.2HCl

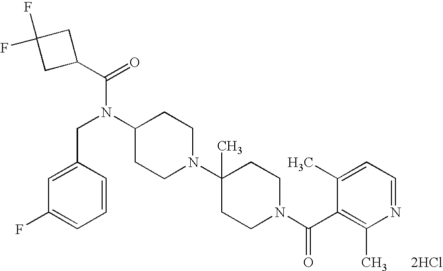

To a solution of the compound of preparation 12 (100 mg, 0.18 mmol) in N,N-dimethylformamide (5 mL) were added triethylamine (102 μL, 0.73 mmol), 3,3-difluorocyclobutanecarboxylic acid (J. Org. Chem. 52 (9), 1872, 1987) (30 mg, 0.22 mmol) and O-(1H-benzotriazol-1-yl)-N,N,N′,N′-tetramethyluronium hexafluorophosphate (83 mg, 0.22 mmol). The reaction mixture was stirred at room temperature for 48 hours after which N,N-dimethylformamide was removed in vacuo and the residue treated with saturated sodium hydrogen carbonate solution (10 mL) and dichloromethane (10 mL). The layers were separated and the aqueous portion was extracted with dichloromethane (2×20 mL). The combined organic extracts were washed with brine (10 mL), dried over magnesium sulfate and reduced in vacuo to give the crude material. The product was purified by column chromatography on silica gel using dichloromethane:methanol:0.88 ammonia (97.5:2.5:0.25) as eluent and then dissolved in ethyl acetate (5 mL) and treated with 2M HCl in diethyl ether to give 50 mg (49%) of the title compound as a white solid.

¹H NMR (400 MHz, CD₃OD) δ 1.48 (3H, t), 1.82-2.01 (3H, m), 2.03-2.22 (3H, m), 2.29-2.47 (2H, m), 2.51-2.63 (4H, m), 2.64-2.78 (3H, dd), 2.82 (1H, s), 2.87-2.95 (2H, m), 3.02-3.27 (4H, m), 3.35-3.51 (2H, m), 3.54-3.62 (1H, m), 3.65-3.72 (1H, m), 4.16-4.25 & 4.61-4.70 (3H, 2×m), 4.76-4.83 (1H, m), 6.90-7.09 (3H, m), 7.27-7.42 (1H, m), 7.83-7.88 (1H, m), 8.60-8.62 (1H, m).

LRMS: m/z APCI+557 [MH⁺].

### EXAMPLE 19

### N-{1′-[(2,4-dimethylpyridin-3-yl)carbonyl]-4′-methyl-1,4′-bipiperidin-4-yl}-N-(3-fluorobenzyl)tetrahydro-2H-pyran-4-carboxamide.2HCl

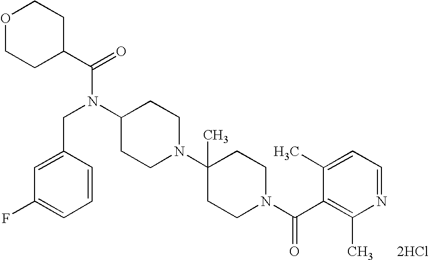

The title compound was prepared according to the method of Example 18 using the compound of preparation 12 and tetrahydro-2H-pyran-4-carboxylic acid (J. Med. Chem. 37, 4538, 1994) to give the title compound as a white solid (25 mg, 25%).

¹H NMR (400 MHz, CD₃OD) δ 1.46-1.50 (3H, m), 1.52-1.82 (2H, m), 1.84-2.01 (4H, m), 2.03-2.22 (4H, m), 2.28-2.39 (2H, m), 2.44-2.49 & 2.67-2.71 (2H, 2×m), 2.51-2.63 (3H, d), 2.65-2.78 (3H, dd), 3.04-3.20 (3H, m), 3.21-3.27 (1H, m), 3.32-3.50 (3H, m), 3.57-3.71 (2H, m), 3.87-4.01 & 4.22-4.47 (2H, 2×m), 4.64-4.78 (3H, m), 6.89-7.11 (3H, m), 7.26-7.43 (1H, m), 7.82-7.87 (1H, m), 8.60 (1H, m).

LRMS: m/z APCI+551 [MH⁺].

### EXAMPLE 20

### N-{1′-[(2,4-dimethylpyridin-3-yl)carbonyl]-4′-methyl-1,4′-bipiperidin-4-yl}-N-(3-fluorobenzyl)tetrahydrofuran-3-carboxamide.2HCl

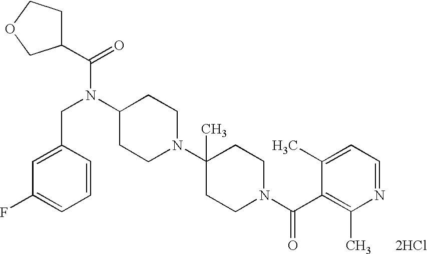

The title compound was prepared from the compound of preparation 12 (100 mg, 0.18 mmol) and (+/−) tetrahydro-3-furoic acid (21 mL, 0.2 mmol) according to the method described above in Example 19, as a white solid in 42% yield.

¹H NMR (400 MHz, CD₃OD) δ 1.46-1.48 (3H, m), 1.88-2.21 (8H, m), 2.24-2.49 (3H, m), 2.51-2.63 (3H, d), 2.65-2.78 (3H, dd), 3.03-3.26 (4H, m), 3.32-3.50 (2H, m), 3.56-3.74 & 3.78-4.10 (5H, 2×m), 4.36-4.79 (4H, m), 6.90-7.11 (3H, m), 7.26-7.43 (1H, m), 7.82-7.87 (1H, m), 8.60 (1H, d).

LRMS: m/Z APCI+537 [MH⁺].

### EXAMPLE 21

### N-benzyl-N-{1′-[(4,6-dimethylpyrimidin-5-yl)carbonyl]-4′-methyl-1,4′-bipiperidin-4-yl}-3,3,3-trifluoropropanamide

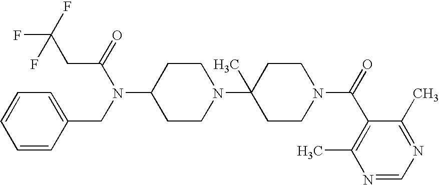

A solution of methyl trifluoroacetate (59 mg, 0.5 mmol), the compound of preparation 15 (100 mg, 0.2 mmol), 1-hydroxybenzotriazole hydrate (62 mg, 0.5 mmol), 1-(3-dimethylaminopropyl)-3-ethylcarbodiimide hydrochloride (88 mg, 0.5 mmol) and triethylamine (96 μL, 0.7 mmol) in dichloromethane (10 mL) was stirred for 24 hours. The reaction mixture was diluted with saturated sodium hydrogen carbonate solution and the organic layer separated and concentrated in vacuo. Purification by column chromatography on silica gel using dichloromethane:methanol (100:0-95:5) as eluent gave 25 mg (61% [symbol] compound as an oil.

¹H NMR (400 MHz, CD₃OD) δ 1.08 (3H, s), 1.30-1.57 (1H, m), 1.60-1.70 (1H, m), 1.72-2.00 (4H, m), 2.03-2.16 (1H, m), 2.30-2.40 & 2.41-2.50 (9H, 2×m), 2.63-3.06 (4H, m), 3.10-3.20 (1H, s), 3.22-3.37 (2H, m), 3.5-3.63 (1H, m), 3.90-4.02 (1H, m), 4.50 (1H, m), 4.58-4.65 (1H, m), 7.10-7.38 (5H, m), 8.90 (1H, m).

LRMS: m/z APCI+532 [MH⁺].

### EXAMPLE 22

### N-benzyl-N-{1′-[(4,6-dimethylpyrimidin-5-yl)carbonyl]-4′-methyl-1,4′-bipiperidin-4-yl}-3,3-difluorocyclobutanecarboxamide

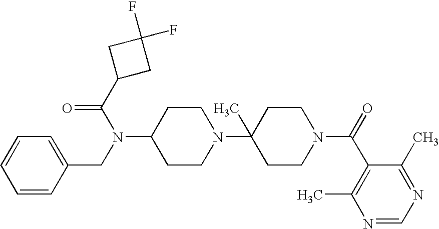

Oxalyl chloride (40 mL, 0.5 mmol) was added dropwise to a stirred solution of 3,3-difluorocyclobutanecarboxylic acid (J. Org. Chem. 52 (9), 1872, 1987) (62 mg, 0.5 mmol) at 0° C. in N,N-dimethylformamide (10 μL, 0.46 mmol) and dichloromethane (20 mL). After addition was complete the reaction mixture was warmed to room temperature and stirred for an hour and then concentrated in vacuo. A solution of the crude material in dichloromethane (20 mL) was added dropwise to a stirred solution of the compound of preparation 15 (100 mg, 0.2 mmol) in dichloromethane (20 mL) and triethylamine (36 μL, 0.7 mmol). The reaction mixture was diluted with saturated sodium hydrogen carbonate solution and the organic layer was separated and dried over magnesium sulfate. Purification by column chromatography on silica gel using dichloromethane:methanol (100:0-95:5) as eluent afforded 22 mg (18%) of the title compound as an oil.

¹H NMR (400 MHz, CD₃OD) δ 0.90 (3H, m), 1.20-1.30 (1H, m), 1.20-1.80 (7H, m), 1.86-2.02 (1H, m), 2.19-2.22 (2H, m), 2.30-2.50 (7H, m), 2.70-3.00 (7H, m), 3.20-3.30 (1H, m), 3.38-3.44 & 4.05-4.17 (1H, 2×m), 4.40-4.60 (3H, m), 7.10-7.40 (5H, m), 8.90 (1H, s).

LRMS: m/z APCI+540 [MH⁺].

### EXAMPLE 23

### N-{1′-[(4,6-dimethylpyrimidin-5-yl)carbonyl]-4′-methyl-1,4′-bipiperidin-4-yl}-3,3,3-trifluoro-N-(2-fluorobenzyl)propanamide

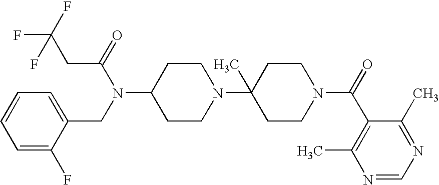

A mixture of 3,3,3-trifluoropropionic acid (177 mg, 1.4 mmol), compound of preparation 13 (101 mg, 0.2 mmol), 1-hydroxybenzotriazole hydrate (186 mg, 1.4 mmol), 1-(3-dimethylaminopropyl)-3-ethylcarbodiimide hydrochloride (264 mg, 1.4 mmol) and triethylamine (288 μL, 1.8 mmol) in dichloromethane (20 mL) was stirred at room temperature for 24 hours. The reaction mixture was diluted with 1M sodium hydroxide solution (20 mL) and the organic layer separated. Concentration in vacuo followed by drying over magnesium sulfate gave the crude residue. Purification by column chromatography on silica gel using dichloromethane:methanol (100:0-95:5) as eluent gave 82 mg (65%) of the title compound as an oil.

¹H NMR (400 MHz, CDCl₃) δ 0.90 (3H, s), 1.20-1.30 (1H, m), 1.30-1.80 (7H, m), 1.80-1.97 (1H, m), 2.05-2.20 (2H, m), 2.40 (6H, d), 2.70-3.00 (3H, m), 3.02-3.18 (1H, m), 3.20-3.50 (3H, s), 4.05-4.20 (1H, m), 4.50 (1H, s), 4.61 (1H, s), 7.00-7.30 (4H, m), 8.90 (1H, s).

LRMS: m/z APCI+550 [MH⁺].

### EXAMPLE 24

### N-{1′-[(4,6-dimethylpyrimidin-5-yl)carbonyl]-4′-methyl-1,4′-bipiperidin-4-yl}-3,3,3-trifluoro-N-(3-fluorobenzyl)propanamide

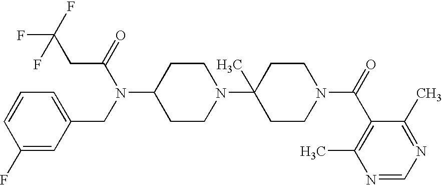

The title compound was prepared from 3,3,3-trifluoropropionic acid (177 mg, 1.4 mmol) and the compound of preparation 13 (101 mg, 0.2 mmol) according to the method described above in Example 23, as an oil in 69% yield.

¹H NMR (400 MHz, CDCl₃) δ 0.90 (3H, s), 1.38-1.45 (1H, m), 1.60-1.82 (7H, m), 1.84-1.98 (1H, m), 2.10-2.15 (2H, m), 2.38-2.50 (6H, m), 2.78-2.85 (1H, m), 2.98-3.03 (3H, m), 3.20-3.50 (2H, m), 4.08 (1H, m), 4.42-4.60 (3H, m), 6.80-7.03 (3H, m), 7.30-7.38 (1H, m), 8.90 (1H, m).

LRMS: m/z APCI+550 [MH⁺].

### EXAMPLE 25

### N-{1′-[(4,6-dimethylpyrimidin-5-yl)carbonyl]-4′-methyl-1,4′-bipiperidin-4-yl}-3,3-difluoro-N-(2-fluorobenzyl)cyclobutanecarboxamide

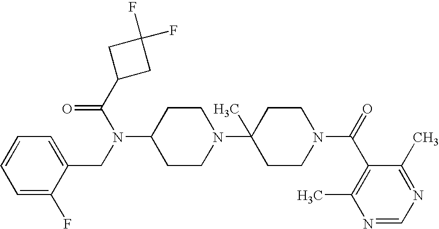

The title compound was prepared from 3,3-difluorocyclobutanecarboxylic acid (J. Org. Chem. 52 (9), 1872, 1987) (47 mg, 0.4 mmol) and the compound of preparation 13 (101 mg, 0.2 mmol) according to the method described above in Example 23, as an oil, 21 mg (16%).

¹H NMR (400 MHz, CDCl₃) δ 0.90 (3H, m), 1.20-1.30 (1H, m), 1.43-1.77 (7H, m), 1.90-2.00 (1H, m), 2.06-2.10 (2H, m), 2.40 (6H, m), 2.70-3.00 (6H, m), 3.10-3.58 (3H, m), 4.04-4.10 (1H, m), 4.50 (2H, m), 4.60 (1H, m), 7.97-7.10 (3H, m), 7.20-7.30 (1H, m), 8.90 (1H, s).

LRMS: m/z APCI+558 [MH⁺].

### EXAMPLE 26

### N-[1′-(2,6-dimethylbenzoyl)-4′-methyl-1,4′-bipiperidin-4-yl]-3,3-difluoro-N-(3-fluorobenzyl)cyclobutanecarboxamide

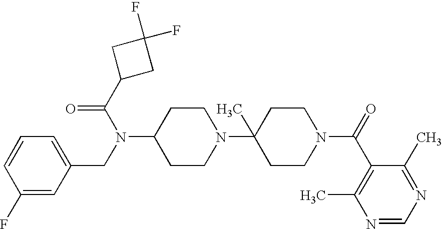

A solution of the compound of preparation 6 (63 mg, 0.2 mmol), 2,6-dimethylbenzoic acid (90 mg, 0.6 mmol), 1-(3-dimethylaminopropyl)-3-ethylcarbodiimide hydrochloride (115 mg, 0.6 mmol), 1-hydroxybenzotriazole hydrate (81 mg, 0.6 mmol) and triethylamine (103 μL, 0.7 mmol) in dichloromethane (20 mL) were stirred at room temperature for 24 hours. The reaction mixture was diluted with saturated sodium hydrogen carbonate solution (10 mL) and the organic layer separated and concentrated in vacuo. Purification by column chromatography on silica gel using dichloromethane:methanol (100:0-95:5) afforded the title compound as an oil, 63 mg (76%).

¹H NMR (400 MHz, CDCl₃) δ 0.97 (3H, m), 1.40-1.50 (1H, m), 1.50-1.90 (8H, m), 1.90-1.98 (1H, m), 2.08-2.20 (2H, m), 2.40-2.50 (6H, m), 2.70-2.90 (4H, m), 2.90-3.00 (3H, m), 3.12-3.17 (1H, m), 3.40-3.58 (1H, m), 4.07-4.18 (1H, m), 4.20-4.50 (2H, m), 4.56-4.59 (1H, m), 6.80-7.00 (3H, m), 7.10-7.20 (3H, m).

LRMS: m/z APCI+556 [MH⁺].

### EXAMPLE 27

### N-[1′-(3,5-dichloroisonicotinoyl)-4′-methyl-1,4′-bipiperidin-4-yl]-3,3-difluoro-N-(3-fluorobenzyl)cyclobutanecarboxamide

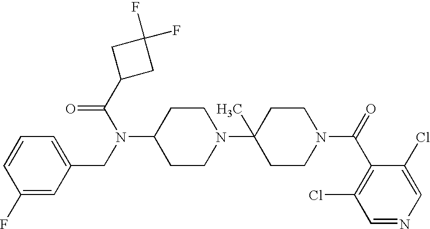

The title compound was prepared from the compound of preparation 6 (63 mg, 0.1 mmol) and 3,5-dichloroisonicotinic acid (115 mg, 0.6 mmol) according to the method described above in Example 26, as an oil in 69% yield.

¹H NMR (400 MHz, CDCl₃) δ 0.95-1.00 (3H, m), 1.23-1.82 (9H, m), 1.90-2.00 (1H, m), 2.10-2.30 (2H, m), 2.40-2.58 (1H, m), 2.70-3.02 (5H, m), 3.30-3.60 (3H, m), 4.08-4.18 (1H, m), 4.40-4.60 (2H, m), 6.80-7.00 (2H, m), 7.20-7.38 (2H, m), 8.50 (2H, m).

LRMS: m/z APCI+598 [MH⁺].

### EXAMPLE 28

### N-{1′-[(2,4-dimethyl-1-oxidopyridin-3-yl)carbonyl]-4′-methyl-1,4′-bipiperidin-4-yl}-3,3-difluoro-N-(3-fluorobenzyl)cyclobutanecarboxamide

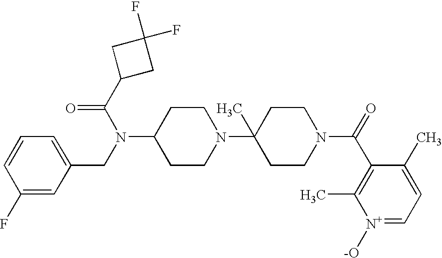

The title compound was prepared from the compound of preparation 6 (63 mg, 0.1 mmol), 2,4-dimethylnicotinic acid 1-oxide (WO2003033490A1, p. 6) according to the method described above in Example 26, as an oil in 90% yield.

¹H NMR (400 MHz, CDCl₃) δ 0.97 (3H, m), 1.21-1.83 (8H, m), 2.20 (3H, m), 2.38 (3H, s), 2.70-3.00 (8H, m), 3.10-3.60 (6H, m), 4.50 (2H, m), 6.80-7.00 (4H, m), 7.30-7.36 (1H, m), 8.10-8.18 (1H, m).

LRMS: m/z APCI+573 [MH⁺].

### EXAMPLE 29

### N-benzyl-N-{1′-[(4,6-dimethylpyrimidin-5-yl)carbonyl]-4′-methyl-1,4′-bipiperidin-4-yl}methanesulfonamide

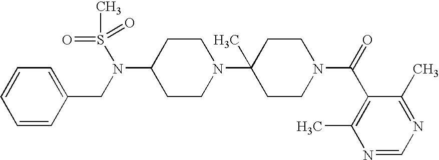

Methanesulfonyl chloride (22 μL, 0.3 mmol) was added dropwise to a stirred solution of the compound of preparation 15 (80 mg, 0.2 mmol) and triethylamine (79 μL, 0.6 mmol) in dichloromethane (10 mL) at room temperature. The reaction mixture was stirred for 30 minutes and diluted with saturated sodium hydrogen carbonate solution (10 mL). The organic layer was separated and concentrated in vacuo and the crude mixture was purified by column chromatography on silica gel using dichloromethane:methanol (100:0-94:6) as eluent to give 84 mg (89%) of the title compound as an oil.

¹H NMR (400 MHz, CDCl₃) δ 0.87 (3H, s), 1.16-1.30 (1H, m), 1.32-1.42 (1H, m), 1.62-1.78 (6H, m), 1.82-1.96 (1H, m), 2.05-2.18 (2H, m), 2.40 (6H, m), 2.77 (3H, m), 2.90 (2H, m), 3.20 (1H, m), 3.36 (1H, m), 3.70 (1H, m), 4.10 (1H, m), 4.38 (2H, m), 7.20-7.40 (5H, m), 8.90 (1H, m).

LRMS: m/z APCI+500 [MH⁺].

### EXAMPLE 30

### N-{1′-[(4,6-dimethylpyrimidin-5-yl)carbonyl]-4′-methyl-1,4′-bipiperidin-4-yl}-N-(2-fluorobenzyl)methanesulfonamide

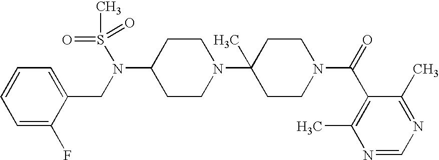

The title compound was prepared from methanesulfonyl chloride (22 μL, 0.3 mmol), the compound of preparation 13 (80 mg, 0.2 mmol) and triethylamine (79 μL, 0.6 mmol) according to the method described above in Example 29, as an oil, 96 mg (98%)

¹H NMR (400 MHz, CDCl₃) δ 0.87 (3H, m), 1.20 (1H, m), 1.97 (1H, m), 1.50-1.80 (6H, m), 1.90 (1H, m), 2.03-2.20 (2H, m), 2.40 (7H, m), 2.80 (3H, m), 2.90 (2H, m), 3.20-3.40 (2H, m), 3.70 (1H, m), 4.40 (2H, m), 6.98 (1H, m), 7.10 (1H, m), 7.20 (1H, m), 7.53 (1H, m), 8.92 (1H, m).

LRMS: m/z APCI+518 [MH⁺].

### EXAMPLE 31

### N-{1′-[(4,6-dimethylpyrimidin-5-yl)carbonyl]-4′-methyl-1,4′-bipiperidin-4-yl}-N-(3-fluorobenzyl)methanesulfonamide

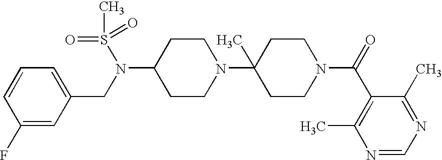

The title compound was prepared from methanesulfonyl chloride (22 μL, 0.3 mmol), the compound of preparation 14 (80 mg, 0.2 mmol) and triethylamine (79 μL, 0.6 mmol) according to the method described above in Example 29, as an oil in 95 mg (85%).

¹H NMR (400 MHz, CDCl₃) δ 0.95 (3H, m), 1.21 (1H, m), 1.40 (1H, m), 1.58 (1H, m), 1.62-1.80 (5H, m), 1.90 (1H, m), 2.10 (2H, m), 2.20 (6H, m), 2.78 (3H, m), 2.93 (2H, m), 3.20 (1H, m), 3.39 (1H, m), 3.70 (1H, m), 4.09 (1H, m), 4.39 (2H, m), 6.95 (1H, m), 7.10 (2H, m), 7.23 (1H, m), 8.90 (1H, m).

LRMS: m/z APCI+518 [MH⁺].

### EXAMPLES 32-53

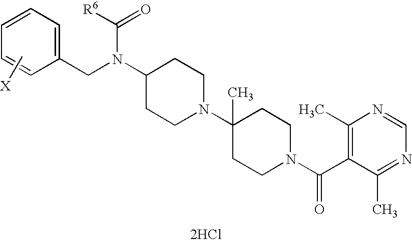

A mixture of the appropriate amine (preparations 13, 14, or 15) (1 eq.), the appropriate acid chloride (R⁶COCl) (1.5 eq.) and triethylamine (3 eq.) in dichloromethane was stirred at room temperature for 3-4 hours. The reaction was washed with sodium hydrogen carbonate solution and the aqueous solution extracted with dichloromethane. The organic solution was separated, dried over magnesium sulfate, concentrated in vacuo and the crude product was purified by column chromatography on silica gel eluting with dichloromethane:methanol (100:0-95:5:0.5). The product was dissolved in a minimum amount of dichloromethane and treated with 2M HCl in diethyl ether to form the title compounds.

| Example | | | LRMS APCI |
|---|---|---|---|
| no. | R⁶ | X | [MH⁺]Data |
| 32 | 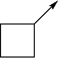 | H | 504 |
| 33 | 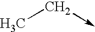 | H | 478 |
| 34 | 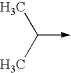 | H | 492 |
| 35 | 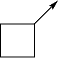 | 2-F | 522 |
| 36 | 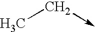 | 2-F | 496 |
| 37 | 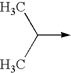 | 2-F | 510 |
| 38 | 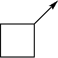 | 3-F | 522 |
| 39 | 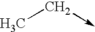 | 3-F | 496 |
| 40 | 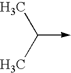 | 3-F | 510 |
| 41 | 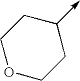 | H | 534 |
| 42 | 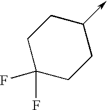 | 2-F | 586 |
| 43 | 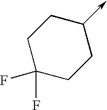 | 3-F | 586 |
| 44 | 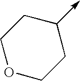 | 3-F | 552 |

### EXAMPLE 45

### N-{1′-[(4,6-dimethylpyrimidin-5-yl)carbonyl]-4′-methyl-1,4′-bipiperidin-4-yl}-N-(2-fluorobenzyl)cyclopropanecarboxamide.2HCl

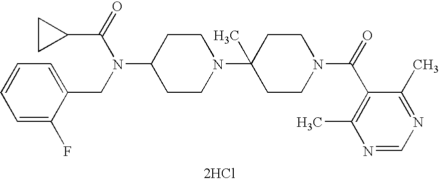

A solution of cyclopropanecarbonyl chloride (31 μL, 0.3 mmol) in dichloromethane (5 mL) was added dropwise to the solution of the compound of preparation 13 (100 mg; 0.2 mmol) and triethylamine (96 μL; 0.7 mmol) in dichloromethane (5 mL). The reaction mixture was stirred at room temperature for 3 hours and then diluted with saturated sodium hydrogen carbonate solution (10 mL). The organic layer was separated, dried over magnesium sulfate and concentrated in vacuo. Purification by column chromatography on silica gel using dichloromethane:methanol:0.88 ammonia (100:0-92:8) The product was dissolved in a minimum amount of dichloromethane and treated with 2M HCl in diethyl ether to afford the title compound, 115 mg (99%).

¹H NMR (400 MHz, CDCl₃) δ 0.62 (2H, m), 0.88 (3H, s), 0.97 (2H, m), 1.18-1.30 (1H, m), 1.38-1.58 (4H, m), 1.59-1.78 (4H, m), 1.90 (1H, m), 2.10-2.22 (1H, m), 2.42 (7H, m), 2.70-2.80 (1H, m), 2.96 (2H, m), 3.30 (1H, m), 3.42 (1H, m), 3.97-4.10 (1H, m), 4.70 (2H, m), 6.90-7.30 (4H, m), 8.90 (1H, m).

LRMS: m/z APCI+508 [MH⁺].

Elemental Analysis: Observe 57.81 (C %), 7.14 (H %), 11.19 (N %); calc for 2HCl.1.5H₂O gives 57.33 (C %), 7.13 (H %), 11.53 (N %).

### EXAMPLE 46

### N-{1′-[(4,6-dimethylpyrimidin-5-yl)carbonyl]-4′-methyl-1,4′-bipiperidin-4-yl}-N-(3-fluorobenzyl)cyclopropanecarboxamide.3HCl

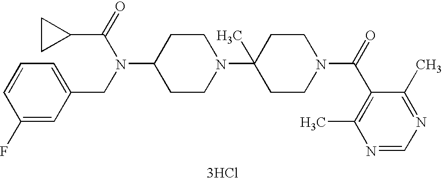

N-{1′-[(4,6-dimethylpyrimidin-5-yl)carbonyl]-4′-methyl-1,4′-bipiperidin-4-yl}-N-(3-fluorobenzyl)cyclopropanecarboxamide was prepared from cyclopropanecarbonyl chloride (31 μL, 0.3 mmol), compound of preparation 13 (100 mg, 0.2 mmol) and triethylamine (96 μL, 0.7 mmol) according to the method described in Preparation 84. The product was dissolved in a minimum amount of dichloromethane and treated with 2M HCl in diethyl ether to afford the title compound, 95 mg (99%).

¹H NMR (400 MHz, CD₃OD) δ 0.62 (2H, m), 0.90 (6H, m), 1.21 (1H, m), 1.28-1.60 (3H, m), 1.61-1.78 (4H, m), 1.85-1.98 (1H m), 2.08-2.20 (2H, m), 2.40 (6H, m), 2.70-2.80 (1H, m), 2.90-3.00 (2H, m), 3.30 (1H, m), 3.43 (1H, m), 4.05 (1H, m), 4.65 (2H, m), 6.80-7.02 (3H, m), 7.18-7.30 (1H, m), 8.80 (1H, s).

LRMS: m/z APCI+508 [MH⁺].

Elemental Analysis: Observe 54.52 (C %), 6.91 (H %), 10.54 (N %); calc for 3HCl.1.3H₂O gives 54.39 (C %), 6.86 (H %), 10.93 (N %).

### EXAMPLE 47

### N-{1′-[(2,4-dimethylpyridin-3-yl)carbonyl]-4′-methyl-1,4′-bipiperidin-4-yl}-N-(3-fluorobenzyl)cyclopropanecarboxamide.2HCl

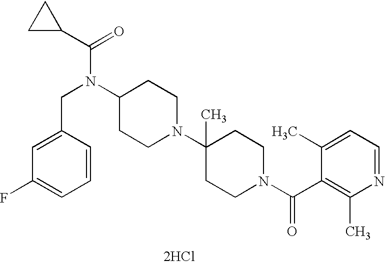

The compound of preparation 12 (100 mg, 0.2 mmol) was suspended in dichloromethane (5 mL) and triethylamine (102 μL, 0.7 mmol) was added. The mixture was cooled to 0° C. and cyclopropanecarbonyl chloride (20 μL, 0.2 mmol) was added dropwise. The reaction mixture was warmed to room temperature and stirred for 72 hours. A solution of saturated sodium hydrogen carbonate (10 mL) was added and the aqueous layer extracted with dichloromethane (10 mL). The combined organic extracts were washed with brine (10 mL), dried over magnesium sulfate and concentrated in vacuo to give the crude residue. Purification by column chromatography on silica gel using dichloromethane:methanol:0.88 ammonia (90:10:1) The product was dissolved in a minimum amount of ethyl acetate and treated with 2M HCl in diethyl ether to afford the title compound, 27 mg (26%).

¹H NMR (400 MHz, CD₃OD) δ 0.82-0.87 (2H, m), 0.94-0.97 (2H, m), 1.74-1.88 (6H, m), 2.11-2.17 (2H, m), 2.32-2.35 (1H, m), 2.54 (3H, m), 2.65 (3H, s), 2.87-2.94 (1H, t), 3.08-3.3.25 (4H, m), 3.32-3.44 (3H, m), 3.56-3.60 (1H, m), 4.85-4.88 (1H, m), 4.99 (2H, s), 7.29-7.33 (5H, m), 7.38-7.42 (2H, m), 9.17 (1H, m).

LRMS: m/z APCI+507 [MH⁺].

### EXAMPLE 48

### N-{1′-[(2,4-dimethylpyridin-3-yl)carbonyl]-4′-methyl-1,4′-bipiperidin-4-yl}-N-(3-fluorobenzyl)cyclobutanecarboxamide.2HCl

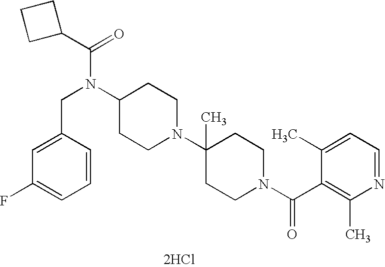

The title compound was prepared according to the method of Example 47 using preparation 12 (100 mg, 0.18 mmol) and cyclobutanecarbonyl chloride, as a white solid (81 mg, 75%).

¹H NMR (400 MHz, CD₃OD) δ 1.47-1.49 (3H, m), 1.80-2.20 (9H, m), 2.25-2.46 (5H, m), 2.51-2.63 (3H, dd), 2.63-2.77 (3H, dd), 3.04-3.22 (3H, m), 3.27-3.70 (5H, m), 4.11-4.20 & 4.55-4.68 (3H, 2×m), 4.77-4.82 (1H, m), 6.90-7.09 (3H, m), 7.26-7.41 (1H, m), 7.81-7.86 (1H, m), 8.60-8.62 (1H, m).

LRMS: m/z APCI+521 [MH⁺].

### EXAMPLE 49

### N-benzyl-N-{1′-[(2,4-dimethylpyridin-3-yl)carbonyl]-4′-methyl-1,4′-bipiperidin-4-yl}cyclobutanecarboxamide

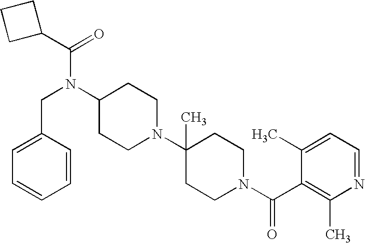

Triethylamine (75 μL, 0.5 mmol) and cyclobutanecarbonyl chloride (31 μL, 0.3 mmol) were added dropwise to a solution of the compound of preparation 10 in dichloromethane (5 mL) at room temperature. The reaction mixture was allowed to stir for two hours and diluted by the addition of saturated sodium hydrogen carbonate solution (5 mL). The phases were separated and the aqueous layer was extracted with dichloromethane (2×10 mL) and the combined organic extracts were dried over magnesium sulfate and concentrated in vacuo. Purification by column chromatography on silica gel using dichloromethane:methanol:0.88 ammonia (95:5:0.5) afforded the title compound, 52.2 g (59%) as a white foam.

¹H NMR (400 MHz, CD₃OD) δ 0.95-1.01 (3H, m), 1.33-2.46 (22H, m), 2.86-2.96 (1H, m), 2.98-3.10 (2H, m), 3.20-3.36 (1H, m), 3.52-3.62 (1H, m), 3.62-3.72 & 4.29-4.40 (2H, 2×m), 3.96-3.85 (1H, m), 4.51-4.61 (2H, m), 7.13-7.29 (5H, m), 7.31-7.38 (1H, m), 8.27-8.33 (1H, m).

LRMS: m/z APCI+503 [MH⁺].

### EXAMPLE 50

### N-benzyl-N-{1′-[(2,4-dimethyl-1-oxidopyridin-3-yl)carbonyl]-4′-methyl-1,4′-bipiperidin-4-yl}-3,3-difluorocyclobutanecarboxamide.HCl

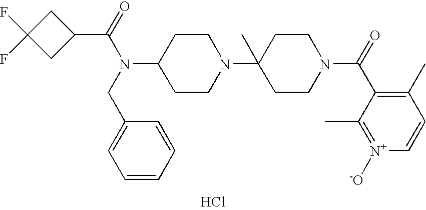

2,4-Dimethyl-1-oxy-nicotinic acid (74 mg, 0.4 mmol), the compound of Preparation 42 (150 mg, 0.3 mmol), 3-(diethoxyphosphoryloxy)-1,2,3-benzotriazin-4(3H)-one (132 mg, 0.4 mmol) and triethylamine (0.16 mL, 1.2 mmol) were dissolved in dichloromethane and stirred at room temperature for 24 hours. The reaction was quenched by the addition of saturated sodium hydrogen carbonate solution and extracted using dichloromethane. The combined organic extracts were concentrated in vacuo to give the crude product. The crude mixture was purified by column chromatography on silica gel using dichloromethane:methanol (100:0 to 90:10) as eluent. The resulting product was then dissolved in dichloromethane (5 mL) and treated with 2M hydrochloric acid in diethyl ether (5 mL), the solvents were removed in vacuo to give 23 mg of the title compound as a white solid.

¹H NMR (400 MHz CDCl₃) δ 0.90 (3H, s), 1.20-1.85 (10H, m), 1.95 (1H, bs), 2.05-2.35 (4H, m), 2.35-2.65 (4H, m), 3.70-3.15 (6H, m), 3.15-3.70 (2H, m), 4.00-4.25 (1H, m), 4.40-4.70 (2H, m), 7.00 (1H, bs), 7.15-7.45 (5H, m), 8.15 (1H, bs).

LRMS: m/z APCI+555[MH⁺]

### EXAMPLE 51

### N-benzyl-N-{1′-[(2,4-dimethylpyridin-3-yl)carbonyl]-4′-methyl-1,4′-bipiperidin-4-yl}propanamide.2HCl

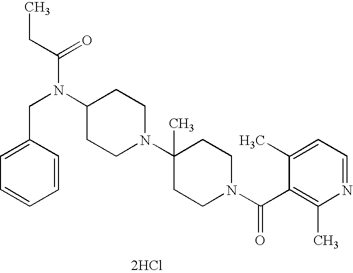

The title compound was prepared from the compound of preparation 10 (75 mg, 0.2 mmol) and propionyl chloride (23 μL, 0.3 mmol) according to the method described above in Example 50, as a white solid in 25% yield.

¹H NMR (400 MHz, CD₃OD) δ 0.95-1.00 (3H, m), 1.01-1.08 (3H, m), 1.16-1.22 (3H, m), 1.33-1.46 (1H, m), 1.48-1.82 (6H, m), 1.85-1.98 (1H, m), 2.13-2.31 & 2.60-2.69 (6H, m), 2.39-2.45 (1H, m), 2.87-2.96 (1H, m), 2.99-3.13 (2H, m), 3.28-3.38 (1H, m), 3.62-3.73 (1H, m), 3.83-3.96 & 4.37-4.48 (2H, 2×m), 4.58-4.67 (2H, m), 7.12-7.30 (5H, m), 7.32-7.40 (1H, m), 8.27-8.33 (1H, m).

LRMS: m/z APCI+477 [MH⁺].

Elemental Analysis: Observe 60.17 (C %), 7.76 (H %), 12.38 (N %); calc for 2HCl. 0.5H₂O gives 60.10 (C %), 7.57 (H %), 12.52 (N %).

### EXAMPLE 52

### N-benzyl-N-{1′-[(2,4-dimethylpyridin-3-yl)carbonyl]-4′-methyl-1,4′-bipiperidin-4-yl}-2-methylpropanamide.2HCl

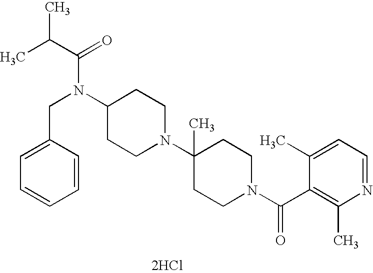

The title compound was prepared from the compound of preparation 10 (75 mg, 0.2 mmol) and isobutyryl chloride (28 μL, 0.3 mmol) according to the method described above in Example 50, as a white solid in 73% yield.

¹H NMR (400 MHz, CD₃OD) δ 0.95-1.01 (3H, m), 1.02-1.23 (4H, m), 1.34-1.45 (1H, m), 1.48-1.82 (6H, m), 1.86-1.97 (1H, m), 2.13-2.33 (7H, m), 2.39-2.45 (3H, m), 2.54-2.63 (1H, m), 2.87-2.96 (1H, m), 2.99-3.10 (2H, m), 3.26-3.37 (1H, m), 3.62-3.73 (1H, m), 3.76-3.96 & 4.36-4.48 (2H, m), 4.56-4.66 (2H, m), 7.14-7.29 (5H, m), 7.30-7.38 (1H, m), 8.27-8.33 (1H, m).

LRMS: m/z APCI+491 [MH⁺].

Elemental Analysis: Observe 61.30 (C %), 7.92 (H %), 12.11 (N %); calc for 2HCl.1.5H₂O gives 61.30 (C %), 7.70 (H %), 12.33 (N %).

### EXAMPLE 53

### N-{1′-[(2,4-dimethylpyridin-3-yl)carbonyl]-4′-methyl-1,4′-bipiperidin-4-yl}-N-(2-fluorobenzyl)cyclopropanecarboxamide.2HCl

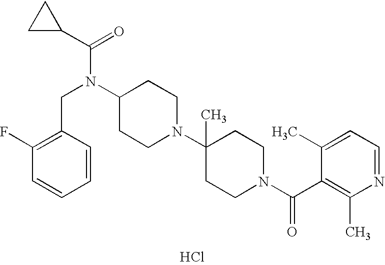

The title compound was prepared from the compound of preparation 11 (100 mg, 0.2 mmol) and cyclopropanecarbonyl chloride (31 μL, 0.3 mmol) according to the method described above in Example 50, as a white solid in 57% yield.

¹H NMR (400 MHz, CDCl₃) 0.80-1.98 (15H, m), 2.12-2.30 (6H, m), 2.40-2.50 (3H, m), 2.70-2.81 (1H, m), 2.90-3.00 (2H, m), 3.20-3.30 (1H, m), 3.40-3.56 (1H, m), 3.90-4.10 & 4.60-4.72 (2H, 2×m), 4.70 (2H, m), 6.90-7.30 (5H, m), 8.30 (1H, s).

LRMS: m/z APCI+507 [MH⁺].

### EXAMPLES 54-83

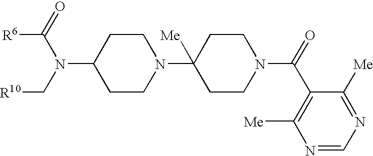

Examples 54 to 83 were prepared by reaction of the title compound of preparation 9 with the corresponding benzylamines R¹⁰CH₂NH₂ and carboxylic acids R⁶CO₂H using the following procedure.

The benzylamines were dissolved as a 0.2M solution in dichloroethane and 170 μl (34 μmol) administered to a 96 well plate. 50 μl (37 μmol) of a 0.74M solution of acetic acid in dichloroethane were added to each well, followed by 300 μl (75 μmol) of a 0.25M suspension of sodium triacetoxyborohydride in dichloroethane and 150 μl (30 μmol) of the compound from preparation 9 as a 0.2M solution in dichloroethane. The plate was sealed and vortexed at room temperature for 48 h. 200 μl of a 4:1 methanol:water mixture was added to each well, vortexing continued for a further hour, and the mixture evaporated to dryness in a Genevac®. The residues were re-dissolved in methanol (400 μl) and purified on Isolute SCX-2 Cartridges® (6 ml tubes, 1 g stationary phase, 0.4 mmol/g loading) using the following method for each tube: 2× condition with 4 ml MeOH, load tube with 500 μl crude, wash each well with 500 μl MeOH, rinse with 2×4 ml MeOH, elute with 5 ml 1 M NH₃ in MeOH.

The N-substituted benzylamine-containing solutions were evaporated to dryness in a Genevac®, dissolved in 150 μl of a 2:1 DMA:triethylamine mixture, and each well treated with 170 μl (51 μmol) of a 0.3M solution of the appropriate carboxylic acid, followed by 250 μl (63 μmol) of a 0.25M solution of HBTU in DMA. The plate was sealed again and heated in to 60° C. for 24 h, allowed to cool to room temperature and the solvent evaporated to dryness in a Genevac®.

The crude title products were dissolved in DMSO (200 μl) and water (150 μl) and each compound purified by preparative HPLC. The purified compounds were characterised by LC-MS analysis.

### Preparative HPLC Conditions:

### Column: Phenomenex Luna® C18, 10 μm, 150×10 mm id

### Temperature: Ambient

Eluent A: 0.05% Diethylamine (aqueous)

### Eluent B: Acetonitrile

Sample solvent: 60% dimethylsulfoxide in water
Initial pump conditions: A % 98, B % 2, flow 8 ml/min
Detection: Gilston® 119 uv detector—225 nm
Injection volume—400 μl

Gradient Timetable

| Time (min) | A% | B% | Flow (ml/min) |
|---|---|---|---|
| 0.0 | 98 | 2 | 8 |
| 0.2 | 98 | 2 | 8 |
| 7.0 | 2 | 98 | 8 |
| 9.0 | 2 | 98 | 8 |
| 9.1 | 98 | 2 | 8 |
| 10.5 | 98 | 2 | 8 |

### LC-MS Conditions

### Column: Phenomenex Luna® C18, 5 μm, 30×4.6 mm id.

### Temperature: 40° C.

Eluent A: 0.05% Diethylamine (aqueous)

### Eluent B: Acetonitrile

Initial pump conditions: A % 90, B % 10, flow 3 ml/min
Injection volume—5 μl
Detection: Start range 210 nm, End range 280 nm, Range interval 5 nm, threshold 0.1 mAU, peakwidth 0.4 min.

Gradient Timetable

| | | | | Pressure |
|---|---|---|---|---|
| Time (min) | A% | B% | Flow (ml/min) | (bar) |
| 0.0 | 90 | 10 | 3 | 400 |
| 2.2 | 5 | 95 | 3 | 400 |
| 2.4 | 5 | 95 | 3 | 400 |
| 2.5 | 90 | 10 | 3 | 400 |

### ELSD: Sedere Dedex 55, Temperature: 40° C., Gas Flow: 2.3 bar

### MS: Platform LC,

ES+ Cone voltage: 26 v, Capillary: 4.08 kv
ES− Cone voltage: −24 v, Capillary: −3.58 kv
Blanket gas: 5001/min, Temperature: 130° C.

| | R⁶ | R¹⁰ | | retention | |
|---|---|---|---|---|---|
| | (arrowhead | (arrowhead | | time | |
| | denotes point of | denotes point of | observed | (ELSD | calc |
| EXAMPLE | attachment) | attachment) | mol ion | trace) | mw |
| 54 | 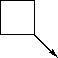 | 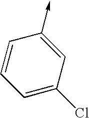 | 538 | 1.99 | 538.132 |
| 55 | 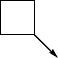 | 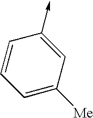 | 518 | 1.87 | 517.714 |
| 56 | 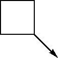 | 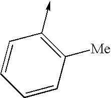 | 518 | 1.9 | 517.714 |
| 57 | 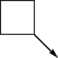 | 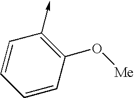 | 534 | 1.85 | 533.713 |
| 58 | 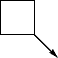 | 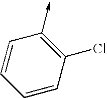 | 538 | 1.96 | 538.132 |
| 59 | 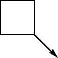 | 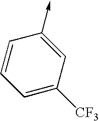 | 572 | 2.08 | 571.684 |
| 60 | 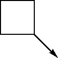 | 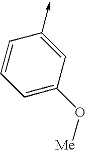 | 534 | 1.74 | 533.713 |
| 61 | 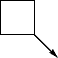 | 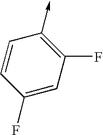 | 540 | 1.87 | 539.667 |
| 62 | 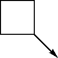 | 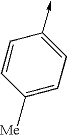 | 518 | 1.88 | 517.714 |
| 63 | 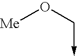 | 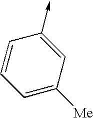 | 508 | 1.48 | 507.675 |
| 64 | 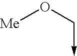 | 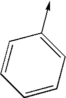 | 494 | 1.35 | 493.648 |
| 65 | 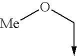 | 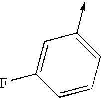 | 512 | 1.43 | 511.638 |
| 66 | 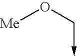 | 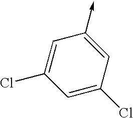 | 562 | 1.81 | 562.538 |
| 67 | 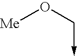 | 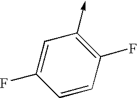 | 530 | 1.46 | 529.628 |
| 68 | 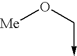 | 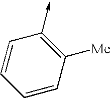 | 508 | 1.43 | 507.675 |
| 69 | 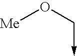 | 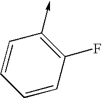 | 512 | 1.4 | 511.638 |
| 70 | 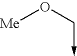 | 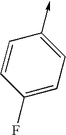 | 512 | 1.42 | 511.638 |
| 71 | 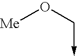 | 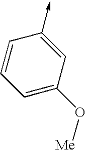 | 524 | 2.06 | 523.674 |
| 72 | 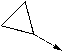 | 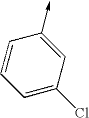 | 524 | 1.82 | 524.105 |
| 73 | 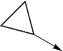 | 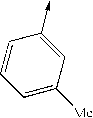 | 504 | 1.69 | 503.687 |
| 74 | 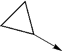 | 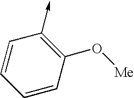 | 520 | 1.7 | 519.686 |
| 75 | 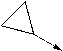 | 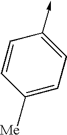 | 504 | 1.78 | 503.687 |
| 76 | 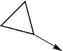 | 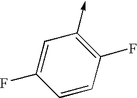 | 526 | 1.78 | 525.64 |
| 77 | 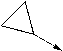 | 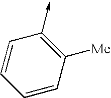 | 504 | 1.65 | 503.687 |
| 78 | 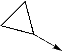 | 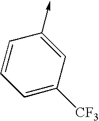 | 558 | 1.89 | 557.657 |
| 79 | 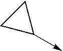 | 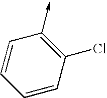 | 524 | 1.76 | 524.105 |
| 80 | 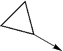 | 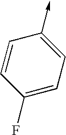 | 508 | 1.69 | 507.65 |
| 81 | 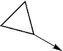 | 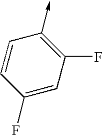 | 526 | 1.78 | 525.64 |
| 82 | 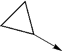 | 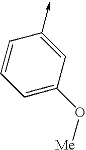 | 520 | 1.57 | 519.686 |
| 83 | 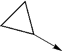 | 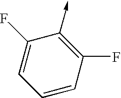 | 526 | 1.6 | 525.64 |

### Preparation 1

### tert-Butyl 4′-cyano-4-hydroxy-1,4′-bipiperidine-1′-carboxylate

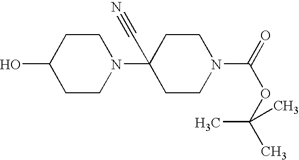

Piperidin-4-ol (10 g, 99 mmol) and 1-BOC-4-piperidone (19.7 g, 99 mmol) were dissolved in dichloromethane (500 mL) at room temperature under N₂. Titanium tetraisopropoxide (29 mL, 109 mmol) was added and the reaction was stirred at room temperature for 18 hours. 1M Diethylaluminium cyanide in toluene (250 mL, 250 mmol) was added, the reaction was stirred for a further 4 hours, then cooled to 0° C. and poured into a mixture of ethyl acetate (1000 mL) and saturated sodium bicarbonate solution (200 mL) at 0° C. This mixture was stirred for 30 minutes during which a thick white precipitate formed. The mixture was filtered through Celite® and the filtrate was washed with water and concentrated in vacuo to give 27.2 g of the title compound as a white solid.

LRMS: m/z APCI+310 [MH⁺].

### Preparation 2

### tert-Butyl 4-hydroxy-4′-methyl-1,4′-bipiperidine-1′-carboxylate

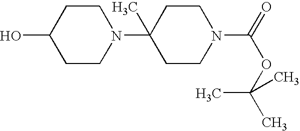

Methylmagnesium bromide (88 mL, 3M in diethyl ether, 264 mmol) was added dropwise to a solution of the compound from preparation 1 (27.2 g, 88 mmol) in tetrahydrofuran (500 mL) at 0° C. under N₂, and once addition was complete the reaction was allowed to warm to room temperature and was stirred for 6 hours. The reaction was cooled to 0° C. and then quenched by the dropwise addition of saturated ammonium chloride solution. The organic layer was separated and the aqueous extracted with ethyl acetate, and the combined organic layers were concentrated in vacuo. The residue was purified by column chromatography on silica gel using dichloromethane:methanol (98:2 to 90:10) as eluent to give 26.1 g (99%) of the title compound as a cream solid.

LRMS: m/z APCI+299 [MH⁺].

### Preparation 3

### tert-Butyl-4′-methyl-4-oxo-1,4′-bipiperidine-1′-carboxylate

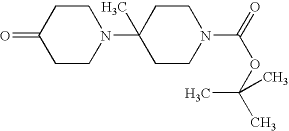

Dichloromethane (200 mL) and dimethylsulfoxide (19 mL, 264 mmol) were added dropwise to a stirred solution of oxalyl chloride (10 mL, 114 mmol) in dichloromethane (300 mL) at −78° C. under N₂. The reaction was stirred for 5 minutes and then the compound from preparation 2 (26.01 g, 88 mmol) in dichloromethane (300 mL) was added dropwise. The reaction was stirred for a further 20 minutes then triethylamine (74 mL, 528 mmol) was added and the mixture allowed to warm to 0° C. over 20 minutes. The reaction was quenched by the addition of water and the organic layer separated. The aqueous phase was extracted with dichloromethane and the combined organic layers concentrated in vacuo. The residue was purified by column chromatography on silica gel using dichloromethane:methanol (100:0 to 97:3) as eluent to give 22.1 g (85%) of the title compound as a yellow oil.

LRMS: m/z APCI+297 [MH⁺].

### Preparation 4

### tert-butyl 4-[(3-fluorobenzyl)amino]-4′-methyl-1,4′-bipiperidine-1′-carboxylate

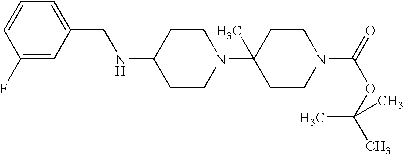

1-(3-Fluorophenyl)methanamine (230 μL, 2.0 mmol), the compound of preparation 3 (300 mg, 1.0 mmol), sodium triacetoxyborohydride (257 mg, 1.2 mmol) and acetic acid (116 μL, 2.0 mmol) were combined and stirred at room temperature for 24 hours. The reaction mixture was then diluted with saturated sodium hydrogen carbonate solution (5 mL), the organic layer was separated and concentrated in vacuo. The crude product was purified by column chromatography on silica gel using dichloromethane:methanol:0.88° ammonia (99:1:0.1) to afford the title compound as a white oil, 385 mg (94%).

LRMS: m/z APCI+406 [MH⁺].

### Preparation 5

### tert-butyl 4-[[(3,3-dimethylcyclobutyl)carbonyl](3-fluorobenzyl)amino]-4′-methyl-1,4′-bipiperidine-1′-carboxylate

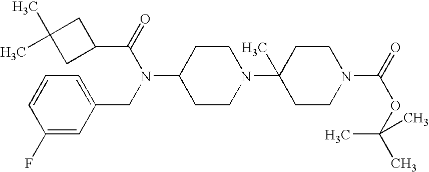

The compound of preparation 4 (385 mg, 1.0 mmol), 3,3-difluorocyclobutanecarboxylic acid (J. Org. Chem. 52 (9), 1872, 1987) (286 mg, 2.1 mmol), 1-hydroxybenzotriazole hydrate (284 mg, 2.1 mmol), 1-(3-dimethylaminopropyl)-3-ethylcarbodiimide hydrochloride (403 mg, 2.1 mmol) and triethylamine (293 μL, 2.1 mmol) in dichloromethane (20 mL) were combined and stirred for 24 hours at room temperature. The reaction mixture was diluted with saturated sodium bicarbonate solution and the organic layer was separated, dried over magnesium sulfate and concentrated in vacuo. Purification by column chromatography on silica gel using dichloromethane:methanol (100:0 to 95:5) afforded the title compound as an oil 219 mg (43%).

LRMS: m/z APCI+524 [MH⁺].

### Preparation 6

### N-(3-fluorobenzyl)-3,3-dimethyl-N-(4′-methyl-1,4′-bipiperidin-4-yl)cyclobutanecarboxamide.HCl

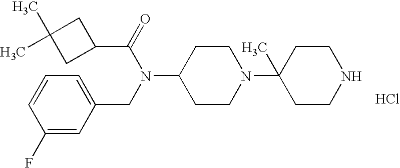

To a solution of compound of preparation 5 (214 mg, 0.4 mmol) in dichloromethane (10 mL) was added 2M hydrochloric acid (20 mL) and the reaction mixture was stirred for 24 hours at room temperature. The solvent was removed in vacuo to afford the title product.

LRMS: m/z APCI+424 [MH⁺].

### Preparation 7

### 4′-Methyl-1,4′-bipiperidin-4-one.HCl

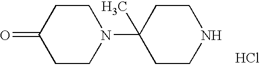

To a cooled solution of compound of preparation 3 (9 g, 30.7 mmol) in diethyl ether (50 mL) was added 1M hydrochloric acid (60 mL) and diethylether (50 mL) and the reaction mixture was stirred for one hour. Removal of solvent afforded the title compound in quantitative yield.

LRMS: m/z APCI+197 [MH⁺].

### Preparation 8

### 1′-[(2,4-Dimethylpyridin-3-yl)carbonyl]-4′-methyl-1,4′-bipiperidin-4-one

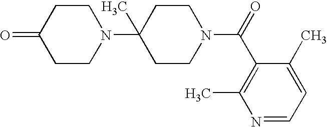

A mixture of the amine hydrochloride of preparation 7 (2.94 g, 10.3 mmol), 2,4-dimethyl-3-carboxypyridine (J. Am. Chem. Soc. 101 (23), 7036, 1979) (1.5 g, 9.93 mmol), 1-(3-dimethylaminopropyl)-3-ethylcarbodiimide hydrochloride (2.9 g, 14.9 mmol), N-ethyldiisopropylamine (7 mL, 39.7 mmol) and 1-hydroxybenzotriazole hydrate (2 g, 14.9 mmol) in dichloromethane (70 mL) was stirred for 72 hours at room temperature. The reaction was diluted with saturated sodium hydrogen carbonate solution (20 mL) and the layers separated. The aqueous layer was extracted using dichloromethane (70 mL). The organic layers were combined and dried over magnesium sulfate and concentrated in vacuo. Purification of the residue by column chromatography using dichloromethane:methanol (98:2 to 95:5) as the eluent afforded the title compound as a foam in 67% yield (2.40 g).

LRMS: m/z APCI+331 [MH⁺].

### Preparation 9

### 1′-[(4,6-Dimethylpyrimidin-5-yl)carbonyl]-4′-methyl-1,4′-bipiperidin-4-one

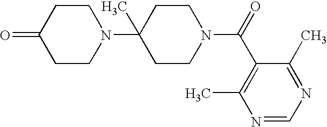

The compound from preparation 3 (9.5 g 32 mmol) in dichloromethane (100 mL) was treated with 2M hydrochloric acid in diethyl ether (40 mL) and stirred at room temperature for 5 hours then the solvent was removed in vacuo. The residue was dissolved in dichloromethane (100 mL) and triethylamine (18 mL, 128 mmol) was added slowly. The solution was stirred for 10 minutes then 4-6-dimethyl-pyrimidine-5-carboxylic acid (U.S. Pat. No. 6,391,865, p. 45) (5.8 g, 38 mmol), 1-hydroxybenzotriazole hydrate (6.5 g, 48 mmol) and 1-(3-dimethylaminopropyl)-3-ethyl carbodiimide hydrochloride (9.2 g, 48 mmol) were added, and the mixture stirred at room temperature for 18 hours. The reaction was quenched with saturated sodium carbonate solution and the organic layer separated and concentrated in vacuo. The residue was purified by column chromatography on silica gel using, ethyl acetate:pentane (50:50 to 80:20) as eluent followed by dichloromethane:methanol (95:5) to give 10.27 g of title compound as a yellow gum.

LRMS: m/z APCI+331 [MH⁺].

### Preparation 10

### N-Benzyl-1′-[(2,4-dimethylpyridin-3-yl)carbonyl]-4′-methyl-1,4′-bipiperidin-4-amine

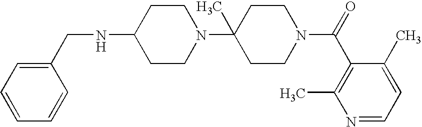

A mixture of the compound of preparation 8 (1.2 g, 3.7 mmol), benzylamine (0.8 mL, 7.2 mmol), sodium triacetoxyborohydride (928 mg, 4.4 mmol), acetic acid (0.42 mL) and dichloromethane (20 mL) were mixed together and stirred for 18 hours at room temperature. The reaction was quenched by the addition of saturated sodium hydrogen carbonate solution and extracted using dichloromethane (3×20 mL). The combined organic extracts were dried over magnesium sulfate and concentrated in vacuo to give the crude product. The crude mixture was purified by flash column chromatography on silica gel using ethyl acetate:methanol:ammonia (90:10:1) and dichloromethane:methanol:0.88 ammonia (90:10:1) to afford the title compound as a white solid in 65% yield (1.00 g).

LRMS: m/z APCI+421 [MH⁺].

### Preparation 11

### 1′-[(2,4-dimethylpyridin-3-yl)carbonyl]-N-(2-fluorobenzyl)-4′-methyl-1,4′-bipiperidin-4-amine

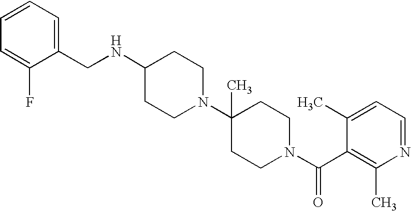

The method of Preparation 10 was used with the compound of preparation 8 (2.3 g, 7 mmol) and 2-fluorobenzylamine to afford the title compound as a white solid (49%, 1.5 g).

LRMS: m/z APCI+439 [MH⁺].

### Preparation 12

### 1′-[(2,4-Dimethylpyridin-3-yl)carbonyl]-N-(3-fluorobenzyl)-4′-methyl-1,4′-bipiperidin-4-amine hydrochloride.HCl

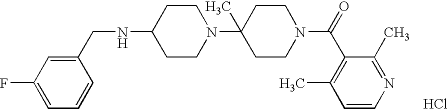

The method of Preparation 10 was used with the compound of preparation 8 (0.86 g, 2.61 mmol) and 3-fluorobenzylamine (0.3 mL, 2.6 mmol) to give 1.38 g of the title compound as a white solid.

LRMS: m/z APCI+439 [MH⁺].

### Preparation 13

### 1′-[(4,6-Dimethylpyrimidin-5-yl)carbonyl]-N-(2-fluorobenzyl)-4′-methyl-1,4′-bipiperidin-4-amine

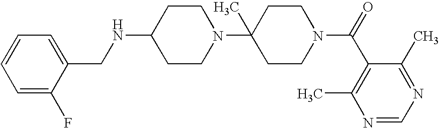

A mixture of the compound from preparation 9 (2.0 g, 6.1 mmol), 2-fluorobenzylamine (1.4 mL, 12.1 mmol), sodium triacetoxyborohydride (1.5 g, 7.3 mmol) and acetic acid (693 μl, 12.1 mmol) in dichloromethane (50 mL) was stirred at room temperature under N₂ for 18 hours. The reaction was then quenched with saturated sodium hydrogen carbonate solution and the organic layer separated and concentrated in vacuo. The resulting solid was triturated with diethyl ether then purified by column chromatography on silica gel using dichloromethane:methanol: 0.88 ammonia (90:10:1) as eluent to give 2.5 g of the title compound as a cream solid.

LRMS: m/z APCI+440 [MH⁺].

### Preparations 14-15

The following compounds of general structure:

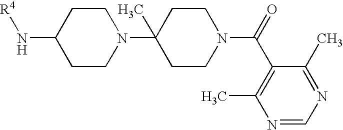

were prepared according to the method described above in Preparation 13.

| Prep | | |
|---|---|---|
| no. | R⁴ | Data |
| 14 | 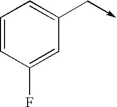 | ¹H NMR (400 MHz, CDCl₃) δ 0.9 (3H, s), 1.2-1.5 (4H, m), 1.8-2.0 (3H, m), 2.0-2.1 (1H, m), 2.1-2.2 (2H, m), 2.4 (3H, s), 2.45 (3H, s), 2.4-2.5 (1H, m), 2.7-7.75 (1H, d), 2.85-3.0 (2H, m), 3.3-3.4 (2H, t), 3.75 (2H, s), 4.25 (1H, br d), 6.9 (1H, dt), 7.0-7.1 (2H, m), 7.2-7.25 (1H, m), 8.9 (1H, s). LRMS: m/z APCI+ 440 [MH⁺] |
| 15 | 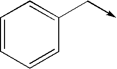 | ¹H NMR (400 MHz, CDCl₃) δ 0.85 (3H, s), 1.11 (1H, m), 1.20 (3H, m), 1.8-2.00 (5H, m), 2.40 (3H, s), 2.5 (3H, s), 2.55 (1H, m), 2.65 (1H, m), 2.82-3.00 (2H, m), 3.38-3.42 (2H, m), 3.80 (2H, s), 4.30 (1H, m), 7.21 (1H, m), 7.30 (5H, m), 8.98 (1H, s). LRMS: m/z APCI+ 422 [MH⁺]. |

### Preparation 16

### tert-butyl 4-(benzylamino)piperidine-1-carboxylate

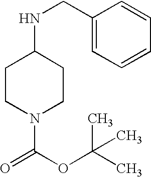

Benzylamine (8.4 mL, 76.8 mmol) was added to a stirring solution of 4-BOC piperidone (15 g, 75.3 mmol) in dichloromethane (225 mL) at room temperature. After 10 minutes, glacial acetic acid (5.4 mL, 94.1 mmol) was added followed by sodium triacetoxyborohydride (23.9 g, 112.9 mmol) after a further 10 minutes. The mixture was stirred for 16 hours. 1M sodium hydroxide solution (50 mL) was added, the layers separated and the organic layer was evaporated under reduced pressure. The aqueous layer was further extracted with dichloromethane (2×50 mL). The combined organic layers were washed with brine, dried over magnesium sulfate and the solvent removed in vacuo to give the title compound as a white solid in 95% yield (20.8 g).

LRMS: m/z APCI+291 [MH⁺].

### Preparation 17

### tert-Butyl 4-[benzyl(cyclopropylcarbonyl)amino]piperidine-1-carboxylate

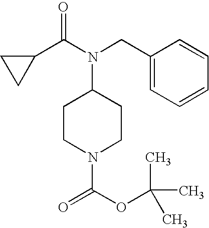

Triethylamine (3.6 mL, 25.8 mmol) was added to a stirring solution of the compound of preparation 16 (5.0 g, 17.2 mmol) in dichloromethane (100 mL) under nitrogen at room temperature. Cyclopropanecarbonyl chloride (1.7 mL, 18.9 mmol) was added and the mixture was stirred for 16 hours. 1M sodium hydroxide solution (20 mL) was added and the organic layer was separated. The aqueous layer was further extracted with dichloromethane (2×25 mL). The combined organic fractions were washed with brine, dried over magnesium sulfate and concentrated in vacuo. The residue was purified by column chromatography on silica gel using 70:30 to 60:40 heptane:ethyl acetate to afford the title compound, 5.8 g (94%).

¹H NMR (400 MHz CDCl₃) δ0.64-0.70 (2H, m), 0.98-1.04 (2H, m), 1.42 (9H, s), 1.36-1.49 (2H, m), 1.49-1.73 (3H, m), 2.64-2.80 (2H, m), 4.01-4.23 (2H, m), 4.66 (2H, s), 4.69 (1H, m), 7.15-7.39 (5H, m).

### Preparation 18

### N-Benzyl-N-piperidin-4-ylcyclopropanecarboxamide

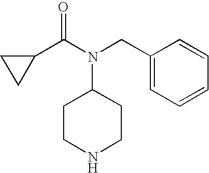

Trifluoroacetic acid (3 mL) was added dropwise to a stirring solution of the compound of preparation 17 (5.7 g, 15.9 mmol) in dichloromethane (30 mL) at 0° C., and the reaction mixture was stirred at room temperature for 16 hours. Further trifluoroacetic acid (6 mL) was added and the mixture was stirred for a further 16 hours. The reaction was quenched by the addition of 1M aqueous sodium hydroxide solution (20 mL), the phases separated and the aqueous layer was extracted with dichloromethane (3×50 mL). The combined organic fractions were washed with brine, dried with magnesium sulfate and concentrated in vacuo to afford the title compound as a white solid, 3.6 g (87% yield).

LRMS: m/z APCI+259 [MH⁺].

### Preparation 19

### tert-Butyl 4-[benzyl(cyclopropylcarbonyl)amino]-4′-cyano-1,4′-bipiperidine-1′-carboxylate

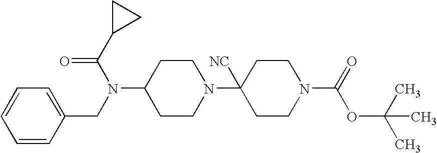

Titanium tetraisopropoxide (3.2 mL, 10.8 mmol) was added to a solution of compound of preparation 18 (2.5 g, 9.8 mmol) and 1-BOC-4-piperidone (395 mg, 2.0 mmol) in dichloromethane (30 mL) under nitrogen at room temperature and stirred for 72 hours. Diethylaluminium cyanide (23.5 mL, 23.5 mmol) (1M in toluene) was added and the mixture stirred for a further 16 hours. The reaction was worked up by adding saturated sodium hydrogen carbonate solution was added (50 mL) followed by ethyl acetate (100 mL). Stirring was continued for 30 minutes and the mixture was filtered through Celite®. The phases were separated and the resulting organic solution was washed with brine, dried over magnesium sulfate and the solvent removed in vacuo to give the title compound as a white solid in quantitative yield.

¹H NMR (400 MHz, CD₃OD) δ 0.70-0.75 & 0.85-1.00 (4H, 2×m), 1.45 (9H, s), 1.60-1.75 (7H, m), 2.10-2.55 (4H, m), 3.00-3.20 (4H, m), 3.85-3.95 (2H, m), 4.20-4.50 (1H, m), 4.60 & 4.80 (2H, s), 7.10-7.40 (5H, m)

LRMS: m/z APCI+467 [MH⁺].

### Preparation 20

### tert-Butyl 4-[benzyl(cyclopropylcarbonyl)amino]-4′-isopropyl-1,4′-bipiperidine-1′-carboxylate

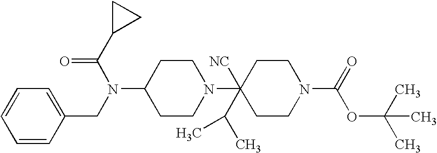

Isopropylmagnesium chloride (6.9 mL, 13.8 mmol) was added to a stirring solution of compound of preparation 19 (2.2 g, 4.6 mmol) in tetrahydrofuran (15 mL) at 0° C. under nitrogen [symbol] ure was allowed to warm to room temperature and stirred for three days. The mixture was quenched by the addition of 1M sodium hydroxide (20 mL) and diluted with ethyl acetate (50 mL). The reaction mixture was filtered through Celite®, washed with brine, dried over magnesium sulfate and concentrated in vacuo. The crude residue was chromatographed on silica gel using dichloromethane:methanol/0.88 ammonia (97.5:2:0.25) as eluent to give 0.5 g (22% yield) of the title compound as a white solid.

LRMS: m/z APCI+484 [MH⁺].

### Preparation 21

### tert-Butyl 4-[benzyl(cyclopropylcarbonyl)amino]-4′-ethyl-1,4′-bipiperidine-1′-carboxylate

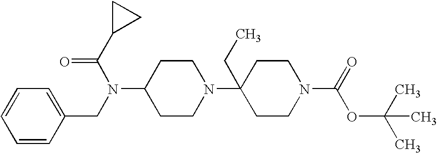

The title compound was prepared from compound of preparation 19 (2.27 g, 4.9 mmol) and ethylmagnesium chloride (7.3 mL, 14.6 mmol) (3M in diethyl ether) according to the method described above in Preparation 20, as a white solid in 44% yield.

LRMS: m/z APCI+470 [MH⁺].

### Preparation 22

### N-benzyl-N-(4′-isopropyl-1,4′-bipiperidin-4-yl)cyclopropanecarboxamide

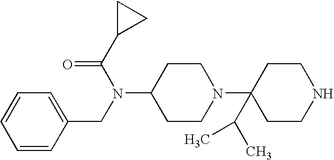

Trifluoroacetic acid (1 mL) was added dropwise to a stirring solution of compound of preparation 20 (0.5 g, 1.1 mmol) in dichloromethane (6 mL) at 0° C. The mixture was allowed to warm to room temperature and stirred for 16 hours. 1M sodium hydroxide solution (30 mL) was added to the mixture which was extracted with dichloromethane (3×50 mL). The combined organic fractions were washed with brine (30 mL), dried over magnesium sulfate and the solvent removed in vacuo to give the title compound as a yellow solid, 0.4 g (97%).

LRMS: m/z APCI+384 [MH⁺].

### Preparation 23

### N-benzyl-N-(4′-ethyl-1,4′-bipiperidin-4-yl)cyclopropanecarboxamide

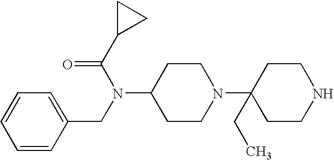

The title compound was prepared from the compound of preparation 21 (0.9 g, 2.0 mmol) and trifluoroacetic acid (2 mL) according to the method described above in Preparation 22, as a yellow solid in quantitative yield.

LRMS: m/z APCI+370 [MH⁺].

### Preparation 24

### N-(1-Benzyl-4-methylpiperidin-4-yl)acetamide

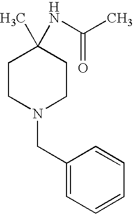

Concentrated sulfuric acid (111 mL) was added dropwise to an ice cold solution of N-(1-benzyl-4-methylpiperidin-4-yl)acetamide (J. Med. Chem. 41 (26), 5320, 1998) (22.8 g, 0.1 mol) in acetonitrile (100 mL) and the reaction mixture was stirred at room temperature for 24 hours. The mixture was then poured into ice (750 mL) and treated with sodium hydroxide until the pH was 10. The solution was then extracted with dichloromethane (3×300 mL). The combined extracts were washed with brine, dried over magnesium sulfate, filtered and concentrated in vacuo to give the title compound as a brown crystalline solid in 52% yield (14.3 g).

LRMS: m/z APCI+247 [MH⁺].

### Preparation 25

### 1-benzyl-4-methylpiperidin-4-amine

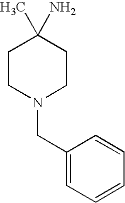

The compound of preparation 24 (14.3 g, 57.8 mol) was heated at reflux in 6M HCl (150 mL) for 8 days. The reaction mixture was cooled to 0° C. and was then treated with 12M sodium hydroxide until the pH was 10. The solution was extracted with ethyl acetate (3×175 mL) and the combined extracts were washed with water (200 mL), brine (200 mL) and dried over magnesium sulfate. The solution was filtered and concentrated in vacuo to afford the title compound as a dark oil in 88% yield (10.5 g).

LRMS: m/z APCI+205 [MH⁺].

### Preparation 26

N,1-dibenzyl-4-methylpiperidin-4-amine

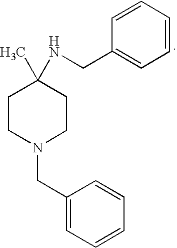

Benzaldehyde (2.6 g, 24.5 mmol) was added to a stirred solution of the compound of preparation 25 (5 g, 24.5 mmol) in dichloromethane (100 mL) under nitrogen. After stirring the reaction mixture for 10 minutes, sodium triacetoxyborohydride (7.3 g, 34.3 mmol) was added and stirring continued for 24 hours. The reaction was quenched by the addition of saturated sodium hydrogen carbonate solution (100 mL) and the organic layer was separated. The aqueous layer was extracted with dichloromethane (50 mL) and the combined organic layers were washed with water (100 mL) and brine (100 mL), dried over magnesium sulfate and concentrated in vacuo to afford the title compound as a dark oil, 6.3 g, (87%).

LRMS: m/z APCI+295 [MH⁺].

### Preparation 27

### N-benzyl-N-(1-benzyl-4-methylpiperidin-4-yl)cyclopropanecarboxamide

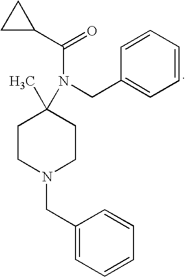

Cyclopropane carbonyl chloride (1.2 mL, 12.8 mmol) was added dropwise to a cooled solution of the compound of preparation 26 (3.1 g, 10.6 mmol) and triethylamine (1.78 mL, 12.8 mmol) in dichloromethane (100 mL) under nitrogen. After stirring for 24 hours, the reaction was diluted with dichloromethane (100 mL) and washed with water (150 mL) followed by sodium hydrogen carbonate solution (150 mL). The organic extract was dried over magnesium sulfate and concentrated in vacuo. Purification by column chromatography on silica gel using dichloromethane:methanol (98:2) as eluent afforded the title compound, 2.3 g (59%).

LRMS: m/z APCI+363 [MH⁺].

### Preparation 28

### N-benzyl-N-(4-methylpiperidin-4-yl)cyclopropanecarboxamide

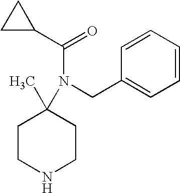

A mixture of the compound of preparation 27 (2.04 g 6.9 mmol), palladium hydroxide (358 mg) and ammonium formate (2.6 g, 41.5 mmol) were heated at 60° C. in ethanol (77 mL) for three hours. The reaction mixture was allowed to cool and then filtered through Arbocel® and the filtrate was concentrated in vacuo. The residue was purified by column chromatography on silica gel using dichloromethane:methanol:0.88 ammonia (90:10:1) as eluent to afford the title compound as an oil, 1.2 g (64%).

LRMS: m/z APCI+273 [MH⁺].

### Preparation 29

### tert-butyl 4-[benzyl(cyclopropylcarbonyl)amino]-4′-isocyano-4-methyl-1,4′-bipiperidine-1′-carboxylate

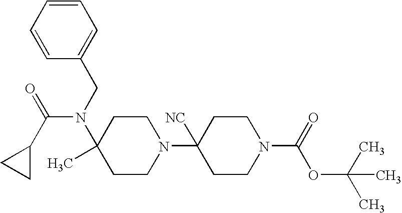

Titanium tetraisopropoxide (643 μL, 2.2 mmol) was added to a stirred solution of compound of preparation 28 (540 mg, 2 mmol) and 1-BOC-4-piperidone (395 mg, 2 mmol) in dichloromethane (10 mL) under nitrogen at room temperature. After stirring the mixture for 24 hours, diethylaluminuim cyanide (4.8 mL, 4.8 mmol), (1M in toluene) was added and stirring continued at room temperature for another 24 hours. The reaction mixture was then diluted with ethyl acetate (30 mL) and treated with saturated sodium hydrogen carbonate solution (20 mL). The mixture was stirred for 15 minutes then filtered through Celite®. The organic layer was separated and washed with brine (30 mL), dried over magnesium sulphate and concentrated in vacuo. The residue was purified by column chromatography using dichloromethane:methanol 0.88 ammonia (95:5:0.5) as eluent to afford the title compound as an oil, 745 mg (78%).

LRMS: m/z APCI+481 [MH⁺].

### Preparation 30

### tert-butyl 4-[benzyl(cyclopropylcarbonyl)amino]-4,4′-dimethyl-1,4′-bipiperidine-1′-carboxylate

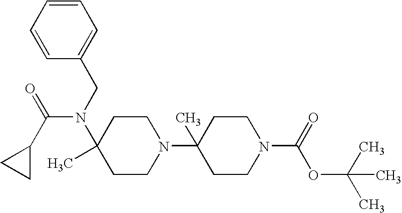

A solution of methylmagnesium bromide (1.5 mL, 4.5 mmol), (3M in diethyl ether) was added dropwise to a stirred solution of the compound of preparation 29 (706 mg, 1.5 mmol) in tetrahydrofuran (12 mL) under nitrogen at 0° C. The mixture was allowed to warm to room temperature and stirred for 24 hours. The reaction was treated with 1M sodium hydroxide solution (40 mL) and diluted with ethyl acetate (40 mL) before filtering through Celite®. The layers were separated and the aqueous extracted with ethyl acetate (2×50 mL). The combined organic layers were washed with brine (50 mL), dried over magnesium sulfate and concentrated in vacuo. The residue was purified by column chromatography on silica gel using dichloromethane:methanol: 0.88 ammonia (95:5:0.25) as eluent to give the title compound as a gum, 481 mg (69%).

LRMS: m/z APCI+470 [MH⁺].

### Preparation 31

### N-benzyl-N-(4,4′-dimethyl-1,4′-bipiperidin-4-yl)cyclopropanecarboxamide.2HCl

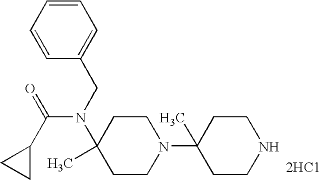

Hydrogen chloride gas was bubbled through a solution of the compound of preparation 30 (481 mg, 1.0 mmol) in ethyl acetate (20 mL) for 10 minutes at room temperature. The mixture was then allowed to stir for 30 minutes and concentrated in vacuo to afford the title compound as a white solid in quantitative yield.

LRMS: m/z APCI+370 [MH⁺].

### Preparation 32

### 3-benzyl-3-azabicyclo[3.2.1]octan-8-one

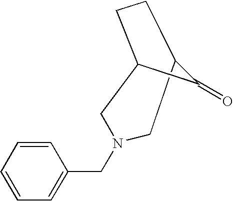

Cyclopentanone (16.8 g, 0.2 mol) was dissolved in acetic acid (300 mL) and benzylamine (28.7 g, 0.2 mol) and formaldehyde (49 mL, 0.6 mol) were added. The mixture was heated to reflux for 5 hours and then allowed to cool to room temperature. The reaction mixture was concentrated in vacuo and diluted with water (150 mL). The aqueous solution was washed with ethyl acetate (2×100 mL) and then basified with solid potassium carbonate. The aqueous layer was extracted with ethyl acetate (3×150 mL). The combined organic extracts were dried over magnesium sulfate and then concentrated in vacuo. The residue was purified by column chromatography on silica gel using pentane:ethyl acetate 80:20 as eluent to give the title compound, 1.36 g (3%) as a white solid.

LRMS: m/z APCI+216 [MH⁺].

### Preparation 33

### N-benzyl-N-{1-[(8-syn)-3-benzyl-3-azabicyclo[3.2.1]oct-8-yl]piperidin-4-yl}cyclopropanecarboxamide

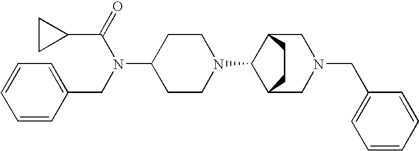

A mixture of the compound of preparation 18 (280 mg, 1.1 mmol) and the compound of preparation 32 (233 mg, 1.1 mmol) in dichloromethane (5 mL) and sodium triacetoxyborohydride (322 mg, 1.5 mmol) was added followed by acetic acid (65 μL, 1.08 mmol). The mixture was stirred at room temperature for 72 hours. The reaction was diluted with 1M sodium hydroxide solution (10 mL), and extracted with dichloromethane (3×20 mL). The combined extracts were washed with brine (10 mL) and dried over magnesium sulfate and then concentrated in vacuo to give the crude residue. Purification by column chromatography using dichloromethane:methanol:0.88 ammonia (97.5:2.5:0.25) gave 259 mg (52%) of the title compound as an oil.

LRMS: m/z APCI+458 [MH⁺].

### Preparation 34

### (8-syn)-N,3-dibenzyl-5-azabicyclo[3.2.1]octan-5-amine

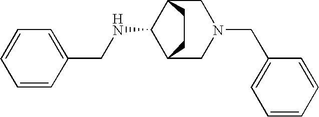

The compound of preparation 32 (400 mg, 1.9 mmol) was dissolved in dichloromethane (10 mL) and benzylamine (205 μL, 1.9 mmol) was added followed by sodium triacetoxyborohydride (551 mg, 2.6 mmol) and acetic acid (110 μL, 1.9 mmol). The mixture was stirred at room temperature under nitrogen for 96 hours. The reaction was diluted with 1M sodium hydroxide solution and extracted with dichloromethane (3×40 mL). The combined extracts were washed with brine (20 mL), dried over magnesium sulfate and concentrated in vacuo. Purification by column chromatography on silica gel using dichloromethane:methanol:0.88 ammonia (98.3:1.3:0.13) as eluent gave the title compound as a light brown oil in quantitative yield.

LRMS: m/z APCI+307 [MH⁺].

### Preparation 35

### N-benzyl-N-[(8-syn)-3-benzyl-3-azabicyclo[3.2.1]oct-8-yl]cyclopropanecarboxamide

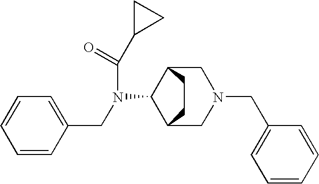

The compound of preparation 34 (490 mg, 1.60 mmol) was dissolved in dichloromethane (20 mL) and triethylamine (670 μL, 4.80 mmol) was added. The mixture was cooled to 0° C. and cyclopropanecarboxylic acid chloride (175 μL, 1.92 mmol) was added dropwise. The reaction mixture was allowed to warm to room temperature and stirred for 48 hours and then diluted with water (20 mL). The mixture was extracted with dichloromethane (3×30 mL) and the combined organic extracts were washed with brine (20 mL), dried over magnesium sulfate and concentrated in vacuo. Purification by column chromatography on silica gel using dichloromethane:methanol:0.88 ammonia (98.3:1.3:0.13) as eluent afforded 137 mg (23%) of the title product as an oil.

LRMS: m/z APCI+375 [MH⁺].

### Preparation 36

### N-{1-[(8-syn)-3-azabicyclo[3.2.1]oct-8-yl]piperidin-4-yl}-N-benzylcyclopropanecarboxamide

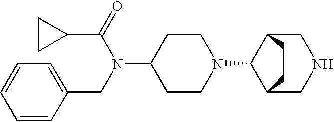

Palladium hydroxide (40 mg) and ammonium formate (182 mg, 2.9 mmol) were added to a solution of the compound of preparation 33 (230 mg, 0.5 mmol) in ethanol (10 mL). The mixture was heated to 60° C. and stirred for 3 hours. The reaction mixture was cooled to room temperature and filtered through Arbocel® washing with ethanol (10 mL). The filtrate was then concentrated in vacuo and purified by column chromatography on silica gel using dichloromethane:methanol:0.88 ammonia (90:10:1) as eluent to give 151 mg (85%) of the title compound as a colourless gum.

LRMS: m/z APCI+368 [MH⁺].

### Preparation 37

### N-[(8-syn)-3-azabicyclo[3.2.1]oct-8-yl]-N-benzylcyclopropanecarboxamide

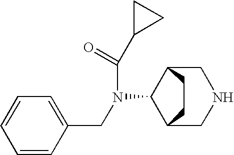

The title compound was prepared from the compound of preparation 35 (230 mg, 0.6 mmol) and palladium hydroxide (50 mg) according to the method described above in Preparation 36, as a colourless gum in 67% yield.

LRMS: m/z APCI+285 [MH⁺].

### Preparation 38

### tert-butyl 4-{(8-syn)-8-[benzyl(cyclopropylcarbonyl)amino]-3-azabicyclo[3.2.1]oct-3-yl}piperidine-1-carboxylate

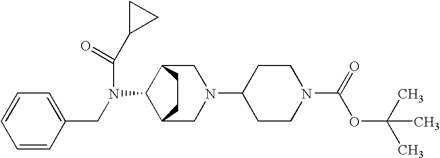

To a solution of the compound of preparation 37 (117 mg, 0.4 mmol) and 1-BOC-4-piperidone (82 mg, 0.4 mmol) in ethanol (20 mL) and was added titanium tetraisopropoxide (182 μL, 0.6 mmol) and the reaction mixture was stirred at room temperature for 48 hours. Sodium cyanoborohydride (39 mg, 0.6 mmol) was then added and the mixture was stirred for a further 7 days at room temperature. The reaction was diluted with ethyl acetate (20 mL) and saturated sodium hydrogen carbonate solution (5 mL) was added. The mixture was stirred vigorously, magnesium sulfate was added and the mixture filtered. The filtrate was then concentrated in vacuo. Purification by column chromatography on silica gel using dichloromethane:methanol:0.88 ammonia (98.3:1.3:0.13) as eluent afforded the title compound as a gum, 127 mg (66%).

LRMS: m/z APCI+468 [MH⁺].

### Preparation 39

### N-benzyl-N-[(8-syn)-3-piperidin-4-yl-3-azabicyclo[3.2.1]oct-8-yl]cyclopropanecarboxamide

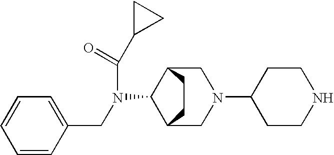

The compound of preparation 38 (120 mg, 0.3 mmol) was dissolved in ethyl acetate (10 mL) and 2M hydrochloric acid in diethyl ether (20 mL) was added and the mixture was stirred for 24 hours at room temperature. The solvent and excess hydrochloric acid were then removed in vacuo. The residue was dissolved in 2M hydrochloric acid (20 mL) and washed with ethyl acetate (2×20 mL). The aqueous layer was basified with solid sodium carbonate and then extracted with ethyl acetate (3×20 mL). The combined organic extracts were washed with brine (20 mL), dried over magnesium sulfate and concentrated in vacuo to afford the title compound as a colourless gum, 51 mg (54%).

LRMS: m/z APCI+368 [MH⁺].

### Preparation 40

### tert-butyl 4-(benzylamino)-4′-methyl-1,4′-bipiperidine-1′-carboxylate

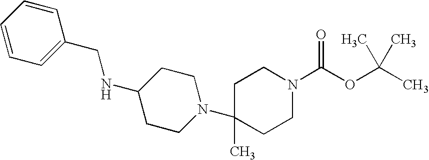

Benzylamine (1.5 mL, 13.5 mmol), the compound of Preparation 3 (2.0 g, 6.7 mmol), sodium triacetoxyborohydride (1.7 g, 8.1 mmol) and acetic acid (0.77 mL, 13.5 mmol) were dissolved in dichloromethane and stirred at room temperature for 24 hours. The reaction was quenched by the addition of saturated sodium hydrogen carbonate solution and extracted using dichloromethane. The combined organic extracts were concentrated in vacuo to give the crude product. The crude mixture was purified by column chromatography on silica gel using pentane:ethyl acetate (0:100 to 100:0) as eluent followed by dichloromethane:methanol (100:0 to 90:10) to give 2.46 g of title compound as a yellow solid.

LRMS: m/z APCI+388[MH⁺]

### Preparation 41

### tert-butyl 4-(benzyl[(3,3-difluorocyclobutyl)carbonyl]amino)-4′-methyl-1,4′-bipiperidine-1′-carboxylate

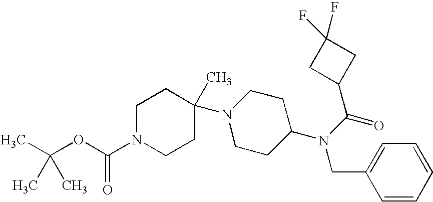

3,3-Difluoro-cyclobutanecarboxylic acid (843 mg, 6.2 mmol), the compound of Preparation 40 (1.6 g, 4.1 mmol), 3-(diethoxyphosphoryloxy)-1,2,3-benzotriazin-4(3H)-one (1.9 g, 6.2 mmol) and triethylamine (1.2 mL, 8.3 mmol) were dissolved in dichloromethane and stirred at room temperature for 24 hours. The reaction was quenched by the addition of saturated sodium hydrogen carbonate solution and extracted using dichloromethane. The combined organic extracts were concentrated in vacuo to give the crude product. The crude mixture was purified by column chromatography on silica gel using pentane:ethyl acetate (0:100 to 100:0) as eluent to give 2.05 g of the title compound as a brown foam.

LRMS: m/z APCI+506[MH⁺]

### Preparation 42

### N-benzyl-3,3-difluoro-N-(4′-methyl-1,4′-bipiperidin-4-yl)cyclobutanecarboxamide.2HCl

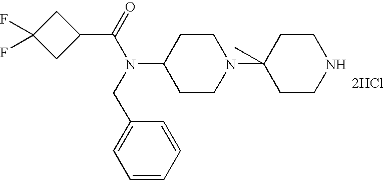

To a solution of compound of Preparation 41 (2.05 g, 4.1 mmol) in methanol (20 mL) was added 2M hydrochloric acid in diethyl ether (30 mL) and the reaction mixture was stirred at room temperature for 24 hours. The solvents were removed in vacuo to give 2.10 g of the title compound as a cream solid.

LRMS: m/z APCI+406[MH⁺]

### Biological Data

The ability of the compounds of formula (I) and their pharmaceutically acceptable salts, solvates and derivatives to modulate chemokine receptor activity is demonstrated by methodology known in the art, such as by using the assay for CCR5 binding following procedures disclosed in Combadiere et al., J. Leukoc. Biol., 60, 147-52 (1996); and/or by using the intracellular calcium mobilisation assays as described by the same authors. Cell lines expressing the receptor of interest include those naturally expressing the receptor, such as PM-1, or IL-2 stimulated peripheral blood lymphocytes (PBL), or a cell engineered to express a recombinant receptor, such as CHO, 300.19, L1.2 or HEK-293.

All the Examples, when tested using the assay for intracellular calcium mobilisation according to Combadiere et al (ibid) were potent antagonists with IC₅₀ values of less than 10 μM.

The pharmacological activity of the compounds of formula (I) and their pharmaceutically acceptable salts, solvates and derivatives is further demonstrated using a gp160 induced cell-cell fusion assay to determine the IC₅₀ values of compounds against HIV-1 fusion. The gp160 induced cell-cell fusion assay uses a HeLa P4 cell line and a CHO-Tat10 cell line.

The HeLa P4 cell line expresses CCR5 and CD4 and has been transfected with HIV-1 LTR-β-Galactosidase. The media for this cell line is Dulbecco modified eagle's medium (D-MEM) (without L-glutamine) containing 10% foetal calf serum (FCS), 2 mM L-glutamine penicillin/streptomycin (Pen/Strep; 100 U/mL penicillin+10 mg/mL streptomycin), and 1 μg/ml puromycin.

The CHO cell line is a Tat (transcriptional trans activator)-expressing clone from a CHO JRR17.1 cell line that has been transfected with pTat puro plasmid. The media for this cell line is rich medium for mammalian cell culture originally developed at Roswell Park Memorial Institute RPMI1640 (without L-glutamine) containing 10% FCS, 2 mM L-glutamine, 0.5 mg/ml Hygromycin B and 12 μg/ml puromycin. The CHO JRR17.1 line expresses gp160 (JRFL) and is a clone that has been selected for its ability to fuse with a CCR5/CD4 expressing cell line.

Upon cell fusion, Tat present in the CHO cell is able to transactivate the HIV-1 long terminal repeat (LTR) present in the HeLa cell leading to the expression of the β-Galactosidase enzyme. This expression is then measured using a Fluor Ace™ β-Galactosidase reporter assay kit (Bio-Rad cat no. 170-3150). This kit is a quantitative fluorescent assay that determines the level of expression of β-galactosidase using 4-methylumbelliferul-galactopyranoside (MUG) as substrate. β-Galactosidase hydrolyses the fluorogenic substrate resulting in release of the fluorescent molecule 4-methylumbelliferone (4MU). Fluorescence of 4-methylumbelliferone is then measured on a fluorometer using an excitation wavelength of 360 nm and emission wavelength of 460 nm.

Compounds that inhibit fusion will give rise to a reduced signal and, following solubilisation in an appropriate solvent and dilution in culture medium, a dose-response curve for each compound can be used to calculate IC₅₀ values.

All the compounds of the Examples of the invention have IC₅₀ values, according to the above method, of less than 25 μM. The compounds of Examples 1, 7, 10, 25, 29, 33, 47, 55 and 78 have, respectively, IC₅₀ values of 13 pM, 1.5 nM, 516 nM, 5.5 nM, 346 nM, 11 nM, 343 pM, 175 nM and 2.5 μM.

1-24. (canceled)

25. The compound of formula (I) 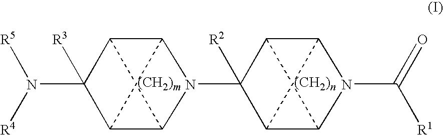
or a pharmaceutically acceptable salt, solvate of derivative thereof, wherein:
R¹ is phenyl; napthyl; or a 5 to 10-membered aromatic heterocycle; wherein said heterocycle contain one to three heteroatoms selected from N, O or S; and wherein the said phenyl, napthyl and heterocycle are substituted by 0 to 3 atoms or groups selected from C₁₋₆ alkyl, C₃₋₇ cycloalkyl, C₁₋₆ alkoxy, C₁₋₆ alkoxy C₁₋₆ alkyl, halogen, haloalkyl, OH, CN, NR⁸R⁹, COR⁸, CO₂R⁸, CONR⁸R⁹, phenyl, imidazolyl, or, wherein R¹ is a heterocycle, oxo;
R² and R³ are independently H or C₁₋₆ alkyl;
R⁴ is benzyl, pyridylmethyl or pyrimidinylmethyl, wherein the said benzyl, pyridylmethyl and pyrimidinylmethyl are substituted by 0 to 3 atoms or groups selected from C₁₋₆ alkyl, C₃₋₇ cycloalkyl, C₁₋₆ alkoxy, C₁₋₆ alkoxy C₁₋₆ alkyl, halogen, C₁₋₆ haloalkyl, OH, CN, NR⁸R⁹, COR⁸, CO₂R⁸, CONR⁸R⁹, phenyl or imidazolyl;
R⁵ is COR⁶ or SO₂R⁷;
R⁶ is C₁₋₆ alkyl, C₃₋₇ cycloalkyl, C₁₋₆ alkoxy, C₁₋₆ alkoxy C₁₋₆ alkyl, tetrahydrofuryl or tetrahydropyranyl; wherein the said C₁₋₆ alkyl, C₃₋₇ cycloalkyl, C₁₋₆ alkoxy and C₁₋₆ alkoxy and C₁₋₆ alkyl are substituted by 0 to 3 atoms or groups selected from halogen, NR⁸R⁹, C₁₋₆ alkoxy or OH;
R⁷ is C₁₋₆ alkyl;
R⁸ and R⁹ are independently H or C₁₋₆ alkyl; or, when R⁸ and R⁹ are both attached to the same N atom, NR⁸R⁹ may also represent a 5 to 7 membered, saturated, partially unsaturated or aromatic, heterocycle containing from 0 to 2 additional heteroatoms selected from O, N or S;
m is 0, 1, 2 or 3;
n is 0, 1, 2 or 3; and wherein
“—” represents an optionally present C—C bond such that, when m or n=1, 2 or 3, any two of the bonds are present per piperidine ring to form an alkylene bridge.

26. The compound as claimed in claim 1 or a pharmaceutically acceptable salt, solvate or derivative thereof, wherein R¹ is phenyl, pyridyl, pyrimidyl, pyridyl N-oxide or pyrimidyl N-oxide; wherein the said phenyl, pyridyl, pyrimidyl, pyridyl n-oxide and pyrimidyl N-oxide are substituted by 0 to 3 atoms or groups selected from C₁₋₆ alkyl, C₃₋₇ cycloalkyl, C₁₋₆ alkoxy, C₁₋₃ alkoxy C₁₋₃ alkyl, halogen, C₁₋₆ haloalkyl, OH, CN, NR⁸R⁹, COR⁸, CO₂R⁸, CONR⁸R⁹, phenyl or imidazolyl.

27. The compound as claimed in claim 1 or a pharmaceutically acceptable salt, solvate or derivative thereof, wherein R¹ is phenyl, pyridyl, pyrimidyl, pyridyl N-oxide or pyrimidyl N-oxide; wherein the said phenyl, pyridyl, pyrimidyl, pyridyl N-oxide and pyrimidyl N-oxide are substituted by 0 to 3 atoms or groups selected from C₁₋₆ alkyl or halogen.

28. The compound as claimed in claim 1 or a pharmaceutically acceptable salt, solvate or derivative thereof, wherein R² and R³ are independently H or C₁₋₃ alkyl.

29. The compound as claimed in claim 1 or a pharmaceutically acceptable salt, solvate or derivative thereof, wherein R⁴ is benzyl substituted by 0 to 3 atoms or groups selected from C₁₋₆ alkyl, C₃₋₇ cycloalkyl, alkoxy, C₁₋₃ alkoxy C₁₋₃ alkyl, halogen, C₁₋₆ haloalkyl, OH, CN, NR⁸R⁹, COR⁸, CO₂R⁸, CONR⁸R⁹, phenyl or imidazolyl.

30. The compound as claimed in claim 29 or a pharmaceutically acceptable salt, solvate or derivative thereof, wherein R⁴ is benzyl substituted by 0 to 3 atoms or groups selected from C₁₋₃ alkyl, C₁₋₃ alkoxy, halogen, or C₁₋₃ haloalkyl.

31. The compound as claimed in claim 25 or a pharmaceutically acceptable salt, solvate or derivative thereof, wherein R⁶ is C₁₋₆ alkyl, C₃₋₆ cycloalkyl, C₃₋₅ cycloalkyC₁₋₂ alkyl, C₁₋₃ alkoxy, C₁₋₃ alkoxy C₁₋₃ alkyl, tetrahydrofuryl or tetrahydropyranyl; wherein the said C₁₋₃ alkyl, C₃₋₆ cycloalkyl, C₃₋₅ cycloalkyl C₁₋₃ alkyl, C₁₋₃ alkoxy and C₁₋₃ alkoxy C₁₋₃ alkyl are substituted by 0 to 3 atoms or groups selected from halogen.

32. The compound as claimed in claim 25 or a pharmaceutically acceptable salt, solvate or derivative thereof, wherein R⁷ is C₁₋₃ alkyl.

33. The compound as claimed in claim 25 wherein R⁸ and R⁹ are independently H or C₁₋₃ alkyl.

34. The compound as claimed in claim 25 having the formula (Ia) 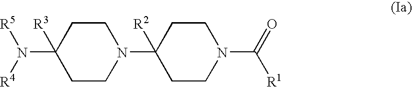
or a pharmaceutically acceptable salt, solvate or derivative thereof.

35. The compound as claimed in claim 25 having the formula (Ib) 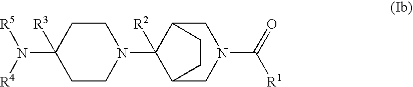
or a pharmaceutically acceptable salt, solvate or derivative thereof.

36. The compound as claimed in claim 25 having the formula (Ic) 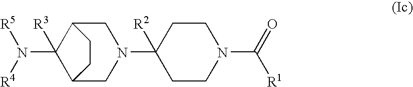
or a pharmaceutically acceptable salt, solvate or derivative thereof.

37. The compound as claimed in claim 25 having the formula (Id) 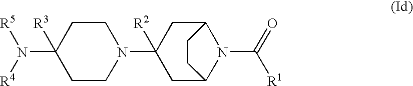
or a pharmaceutically acceptable salt, solvate or derivative thereof.

38. The compound as claimed in claim 25 having the formula (Ie) 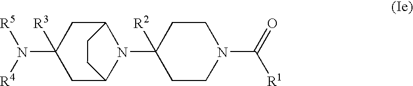
or a pharmaceutically acceptable salt, solvate or derivative thereof.

39. A pharmaceutical composition comprising the compound of formula (I) according to claim 1 or a pharmaceutically acceptable salt, solvate or derivative thereof, together with one or more pharmaceutically acceptable excipients, diluents or carriers.

40. The pharmaceutical composition according to claim 39 including one or more additional therapeutic agents.

41. A method of treatment of a mammal suffering from a disorder in which the modulation of CCR5 receptors is implicated which comprises treating said mammal with an effective amount of the compound of formula (I) according to claim 1 or a pharmaceutically acceptable salt, solvate or derivative thereof.

42. The method of claim 41 wherein the disorder is HIV, a retroviral infection genetically related to HIV, AIDS, an inflammatory disease, an autoimmune disease or pain.

43. The method of claim 41 wherein the disorder is multiple arthritis, Behcet's disease, Sjogren's syndrome, systemic sclerosis or graft rejection.

44. The method of claim 41 wherein the disorder is selected from inflammatory bowel disease, inflammatory lung conditions, endometriosis, renal diseases, fibrosis, encephalitis, chronic heart failure, myocardial infarction, hypertension, stroke, ischaemic heart disease, restenosis, atherosclerotic plaque, obesity, psoriasis, CNS diseases, anaemia, atopic dermatitis, chronic pancreatitis, Hashimoto's thyroiditis, type I diabetes, cancer, pain, or a stress response resulting from surgery, infection, injury or other traumatic insult.

45. The method of claim 41 wherein the disorder is HBV, HCV, plague, pox virus, toxoplasmosis, mycobacterium, trypanosomal, pneumonia, or cytosporidiosis.
